SEQUENCE LISTING (8 sequences)

SEQ ID NO: 1 - DNA, 18 bp, synthetic construct
  source 1..18: mol_type = other DNA
acatgacggaggttgtga

SEQ ID NO: 2 - DNA, 19 bp, synthetic construct
  source 1..19: mol_type = other DNA
cgcaaatttccttccactc

SEQ ID NO: 3 - DNA, 21 bp, synthetic construct
  source 1..21: mol_type = other DNA
gctagcctatctccggctaaa

SEQ ID NO: 4 - DNA, 20 bp, synthetic construct
  source 1..20: mol_type = other DNA
ctggaaaagggtccagatga

SEQ ID NO: 5 - DNA, 20 bp, synthetic construct
  source 1..20: mol_type = other DNA
atgcatggacctaaggcaac

SEQ ID NO: 6 - DNA, 20 bp, synthetic construct
  source 1..20: mol_type = other DNA
aggtcgtctgctgagctttc

SEQ ID NO: 7 - DNA, 19 bp, synthetic construct
  source 1..19: mol_type = other DNA
caaccgcgagaagatgacc

SEQ ID NO: 8 - DNA, 20 bp, synthetic construct
  source 1..20: mol_type = other DNA
agaggcgtacagggatagca

(19) United States
(12) Patent Application Publication
(10) Pub. No.: US 2026/0191819 A1
(43) Pub. Date: Jul. 9, 2026

(54) COMPOSITION FOR THE TREATMENT OF THE HUMAN PAPILLOMAVIRUS INFECTION (HPV)

(71) Applicant: LO.LI. PHARMA S.R.L., Rome (IT)

(72) Inventors: Vittorio UNFER, Rome (IT); Isabella NERI, Rome (IT)

(73) Assignee: LO.LI. PHARMA S.R.L., Rome (IT)

(21) Appl. No.: 18/874,777

(22) PCT Filed: Jun. 27, 2023
(86) PCT No.: PCT/EP2023/067495

§ 371 Date: Dec. 13, 2024

(30) Foreign Application Priority Data
Jun. 30, 2022 (IT) 102022000013867

(51) Int. Cl. A61K 31/353; A61K 31/519; A61K 31/714; A61K 31/728; A61K 36/82; A61P 31/20

(52) U.S. Cl. CPC A61K 31/353; A61K 31/519; A61K 31/714; A61K 31/728; A61K 36/82; A61P 31/20

(57) ABSTRACT

The present invention regards a combination or composition including epigallocatechin gallate (EGCG) and at least one other agent selected from folic acid, vitamin B12 and low or very low molecular weight hyaluronic acid (1-500 kDa), or derivatives, for the prevention, the control, and the treatment of a human papillomavirus infection in a subject.

### FIELD OF INVENTION

The present invention regards a combination or a composition comprising epigallocatechin gallate (EGCG) and at least one the following further agents: folic acid, vitamin B12, low or very low molecular weight hyaluronic acid (1-500 kDa) or derivatives thereof, for the prevention, control and treatment of a human papilloma virus (HPV) infection in a subject.

### BACKGROUND

The human papillomavirus (HPV) is an infection that is globally present in sexually active populations. It is estimated that 80% of people will be affected by this type of infection at least once in their lifetimes. [Sung H, Ferlay J, Siegel R L, Laversanne M, Soerjomataram I, Jemal A, Bray F. Global Cancer Statistics 2020: GLOBOCAN Estimates of Incidence and Mortality Worldwide for 36 Cancers in 185 Countries. CA Cancer J Clin. 2021 May; 71 (3): 209-249. doi: 10.3322/caac.21660]. The HPV infection affects both women and men and is transfected primarily following sexual intercourse (vaginal or anal) and occasionally non-penetrative sexual activities. The viruses of the HPV family have a marked tropism for epithelial tissues and mucous membranes which, during sexual intercourse are subjected to microtraumas and abrasions. Papillomaviruses (from the Latin “papilla” meaning pustules and the Greek “oma” meaning tumour) are a numerous family of double stranded DNA viruses (8 kb). They have no envelope and an icosahedral symmetry. The viral genome of HPV viruses is divided into two defined regions 1) early and 2) late. The early genes code for the early proteins E1, E2, E4, E5, E6, and E7 which are responsible for the replication of viral DNA and for the cellular transformation. The late genes code for the late proteins L1 and L2 which are needed for the formation of the viral capsid [Mclaughlin-Drubin, M. E.; Munger, K. Oncogenic activities of human papillomaviruses. Virus Res. 2009, 143, 195-208]. The HPV viruses are classified by their oncogenicity. Those with a low risk are associated with proliferative lesions of the skin and the mucous membrane, which are generally benign. Clinical manifestations include common, flat, and planar warts, genital and flat condyloma acuminata (anogenital warts) which are the result of sexual transmission of the virus, arising in the penis, anus, and female genitalia. The HPV viruses associated with a high risk are those which integrate within the genome of the host and cause lesions which in time can evolve into carcinomas. The papillomavirus is in fact the main etiological agent of cervical cancer, which is the fourth most common tumour among women and the second between ages 15-14 [Sung H, Ferlay J, Siegel R L, Laversanne M, Soerjomataram I, Jemal A, Bray F. Global Cancer Statistics 2020: GLOBOCAN Estimates of Incidence and Mortality Worldwide for 36 Cancers in 185 Countries. CA Cancer J Clin. 2021 May; 71 (3): 209-249. doi: 10.3322/caac.21660]. The HPV infection is also responsible for 50% of the cases of penile cancer, 33-72% of mouth cancer, and approximately 85% of anal cancer in men [Giuliano A R, Anic G, Nyitray A G. Epidemiology and pathology of HPV disease in men. Gynecol Oncol. 2010; 117 (2 Suppl): S15-S19. doi: 10.1016/j.ygyno.2010.01.026]. The majority of HPV infections are generally transient and resolve spontaneously over the course of 1-2 years. The course of infection from the initial infection to tumour is a long path consisting of many steps. What essentially determines the “point of no return” is the persistence of the virus and its integration into the host (Evander M, Edlund K, Gustafsson A, Jonsson M, Karlsson R, Rylander E, Wadell G. Human papillomavirus infection is transient in young women: a population-based cohort study. J Infect Dis. 1995 April; 171 (4): 1026-30. doi: 10.1093/infdis/171.4.1026). The initial infection occurs when the virus particle enters the cells of the epithelium's basal layer, following a small wound or trauma which allows exposure of the basal layer to the keratinocytes of the virus. (Woodman C B, Collins S I, Young L S. The natural history of cervical HPV infection: unresolved issues. Nat Rev Cancer. 2007 January; 7 (1): 11-22. doi: 10.1038/nrc2050. PMID: 17186016). When the virus colonizes the basal and parabasal cells of the epithelium, the viral genome replicates in an episomal form as an extrachromosomal fragment of circular DNA. In this phase, the expression of viral genes is limited to those involved in stimulating the infected cell to proliferate and expand laterally. During episomal replication the cells are infected with a virus that is functionally inactive (latency phase) and the cells do not show any signs of neoplastic transformation. In this phase of replication, the viral protein E2 has a fundamental role in regulating the levels of E6 and E7, the two primary viral oncoproteins. The loss of E2 represents the fight stage of neoplastic transformation. The E1 & E2 region becomes cleaved when the viral genome is integrated into the host, while the E6/E7 region remains intact. Following this integration, a functional loss in both, the E2 gene and the protein is seen, causing an increased expression in the expression of the viral oncoproteins E6 and E7. These proteins act on cell checkpoints, binding to the onco-suppressive proteins pRB and p53, which act as cell checkpoint regulators. Specifically, pRB is a negative regulator that normally prevents entrance in the S phase of the cell cycle; p53, dubbed as the “guardian of the genome”, is the principal regulator of the cell cycle [Lane D P. Cancer. p53, guardian of the genome. Nature. 1992 Jul. 2; 358 (6381): 15-6. doi: 10.1038/358015a0. PMID: 1614522]. The loss of onco-suppressive function facilitates the onset of the tumour. In fact, the only strategy available to limit the spread of the infection are screening programs to catch the infection early and the vaccination campaign. It must be remembered that the vaccine only influences certain strains of the HPV family, it is not without risks and its efficiency diminishes over time. Currently, no specific treatment exists to prevent or block the infection of HPV.

### Epigallocatechin Gallate (EGCG)

Epigallocatechin gallate (EGCG) is an ester of epigallocatechin and gallic acid, which is extracted from green tea. It's effect in oncology has been studied in depth and it has demonstrated a beneficial effect in diverse tumour types, including cervical cancer. [Wang Y Q, Lu J L, Liang Y R, Li Q S. Suppressive Effects of EGCG on Cervical Cancer. Molecules. 2018 Sep. 12; 23 (9): 2334. doi: 10.3390/molecules23092334. PMID: 30213130; PMCID: PMC6225117]. EGCG shows an anti-proliferative, pro-apoptotic, anti-metastatic, and anti-inflammatory effect on tumour cells and cell lines infected with high-risk HPV. EGCG reduces tumoral proliferation in various ways as: 1) blocking the cell cycle of tumour cells and/or 2) blocking the tumoral proliferation. The study of Changping Zou et al. [Zou C, Liu H, Feugang J M, Hao Z, Chow H H, Garcia F. Green tea compound in chemoprevention of cervical cancer. Int J Gynecol Cancer. 2010; 20 (4): 617-624. doi: 10.1111/IGC.0b013e3181c7ca5c] demonstrated that EGCG inhibits cellular growth and promotes apoptosis, i.e., cell death, in a dose-dependent manner in an epithelial cervical cell line infected with high-risk HPV (TLC1 and cervical cancer lines such as Hela and Me180. Furthermore, EGCG increases the level of p53 protein expression and reduces that of the viral protein E7 [Zou C, Liu H, Feugang J M, Hao Z, Chow H H, Garcia F. Green tea compound in chemoprevention of cervical cancer. Int J Gynecol Cancer. 2010; 20 (4): 617-624. doi: 10.1111/IGC.0b013e3181c7ca5c]. A randomised study demonstrated that a green tea extract containing 200 mg of EGCG had a chemo-preventive effect on cervical cancer. [Ahn W S, Yoo J, Huh S W, Kim C K, Lee J M, Namkoong S E, Bae S M, Lee I P. Protective effects of green tea extracts (polyphenon E and EGCG) on human cervical lesions. Eur J Cancer Prev. 2003 October; 12 (5): 383-90. doi: 10.1097/00008469-200310000-00007]. In this study 88 women with lesions of various grades were treated with EGCG tablets (200 mg) for a period of 8-12 weeks. 60% of treated women saw an improvement of their general clinical status; 100% of women with moderate lesions and 71% of those with serious lesions responded positively to the treatment, following a cytologic evaluation, colposcopy, histological examination, and an HPV DNA test. In addition, EP0842660A1 describes a pharmaceutical composition containing a tea (Camellia sinensis) extract containing catechins (more particularly (−)-epigallocatechin gallate) in the form of a 2-20% by weight ointment or a 50-500 mg tea catechin suppository and its use in the treatment of condyloma acuminata (genital warts) caused by human papilloma virus. CN107349197 discloses application of catechin in preparation of a medicine for treating skin papilloma.

### Folic Acid and Vitamin B12

Vitamin B12 (cobalamin), folic acid and the folate derivatives are essential water-soluble vitamins, fundamental for numerous functions of an organism. Specifically, these two substances are involved in the metabolism of methionine (an amino acid essential for protein synthesis), the synthesis of nucleic acids, red blood cell, and nervous system function. A lack of folate and vitamin B12 causes an increase in plasma homocysteine, which is a risk factor for cardiovascular disease, Alzheimer's, and other neurological disorders. A recent meta-analysis has demonstrated that folate deficiency and a consequent increase in homocysteine increases the risk of cancer. [Zhang D, Wen X, Wu W, Guo Y, Cui W. Elevated homocysteine level and folate deficiency associated with increased overall risk of carcinogenesis: meta-analysis of 83 case-control studies involving 35,758 individuals. PLOS One. 2015 May 18; 10 (5): e0123423. doi: 10.1371/journal.pone.0123423]. Furthermore, folate and vitamin B12 are essential for the synthesis of S-adenosyl methionine (SAM), which is a methyl group donor and an essential cofactor in numerous pathways of biochemical synthesis. Folate and vitamin B12, due to their role in the methylation of DNA, and more specifically in the methylation of the viral genome of HPV, are essential for regulating and inhibition the genetic expression of the virus in addition to, proliferation and persistence. [Sedjo R L, Inserra P, Abrahamsen M, Harris R B, Roe D J, Baldwin S, Giuliano A R. Human papillomavirus persistence and nutrients involved in the methylation pathway among a cohort of young women. Cancer Epidemiol Biomarkers Prev. 2002 April; 11 (4): 353-9]. A study of 315 women with various degrees of high-risk HPV infection saw a correlation with levels of folate and B12 with the level of methylation of the viral genome. This study demonstrated that both folate and vitamin B12 reduced the risk of high-risk lesions, and that high levels of both corresponded with high levels of methylation of the HPV genome and a reduced percentage of high risks legions. [Piyathilake C J, Macaluso M, Chambers M M, Badiga S, Siddiqui N R, Bell W C, Edberg J C, Partridge E E, Alvarez R D, Johanning G L. Folate and vitamin B12 may play a critical role in lowering the HPV 16 methylation-associated risk of developing higher grades of CIN. Cancer Prev Res (Phila). 2014 November; 7 (11): 1128-37. doi: 10.1158/1940-6207.CAPR-14-0143]. The study conducted by Piyathilake et al. validated the protective effect of folate on the occurrence, persistence, and disappearance of high-risk HPV infection. A total of 345 women at risk of developing high-risk legions were monitored for two consecutive years and their clinical status assessed every 6 months, measuring metabolic folate levels. The results of this study demonstrated that the patients with higher folate levels had a 73% reduced probability to test positive during periodic follow up visits; specifically high folate levels reduced the incidence and persistence of the virus, while helping the clearance of the infection. [Piyathilake C J, Henao O L, Macaluso M, Cornwell P E, Meleth S, Heimburger D C, Partridge E E. Folate is associated with the natural history of high-risk human papillomaviruses. Cancer Res. 2004 Dec. 1; 64 (23): 8788-93. doi: 10.1158/0008-5472.CAN-04-2402].

### Low and Very Low Weight Hyaluronic Acid

It is known that hyaluronic acid is an interesting, versatile, and useful natural macromolecule. Despite the simplicity of its structure, it possesses specific properties and has different functions depending on its molecular weight. Physiologically hyaluronic acid is present as a high molecular weight (HMW-HA) salt (sodium hyaluronate) and has a structural role as a component of the extracellular matrix. Furthermore, hyaluronic acid has a regulatory role. Specifically, low and very low weight hyaluronic acid stimulate the production of pro-inflammatory cytokines such as IL-1B, IL-6, IL-12, TNF-α [Jiang D, Liang J, Noble P W. Hyaluronan as an immune regulator in human diseases. Physiol Rev. 2011 January; 91 (1): 221-64. doi: 10.1152/physrev.00052.2009]. The stimulation of these pro-inflammatory factors is extremely useful in the “wound healing” process [Yang H, Song L, Zou Y, Sun D, Wang L, Yu Z, Guo J. Role of Hyaluronic Acids and Potential as Regenerative Biomaterials in Wound Healing. ACS Appl Bio Mater. 2021 Jan. 18; 4 (1): 311-324. doi: 10.1021/acsabm.0c01364]. The study of Gao F. et al [Gao F, Yang C X, Mo W, Liu Y W, He Y Q. Hyaluronan oligosaccharides are potential stimulators to angiogenesis via RHAMM mediated signal pathway in wound healing. Clin Invest Med. 2008; 31 (3): E106-16. doi: 10.25011/cim.v31i3.3467] has demonstrated how low and very low weight hyaluronic acid, in a cell model in vitro, significantly accelerates the process of wound healing in comparison with the control. In the context of HPV infections, this repairing action allows for quicker restoration of the integrity of the epithelium and mucous membranes that undergo trauma during sexual intercourse, which is responsible for allowing access of the virus to the epithelial basal layer. The role of hyaluronic acid and its repairment action have already been demonstrated in the field of vaginal infections in addition to other therapeutic areas. The study of La Galia et al. 2014 [La Galia T., Micali A., Puzzolo D., Cancellieri F. Oral Low-Molecular Weight Hyaluronic Acid in the Treatment of Atrophic Vaginitis. International Journal of Clinical Medicine>Vol. 5 No. 11, May 2014] validated the effect of low and very low weight hyaluronic acid in 12 post-menopausal women with vaginal atrophy. Specifically, the study proved that a 3-month treatment with hyaluronic acid restores the mucous membrane, dramatically reducing symptoms related to vaginal atrophy (itching, burning, and dyspareunia). EP3804696A1 also relates to the treatment of warts which are caused by infection with a type of HPV with patches containing hyaluronic acid.

Within the present invention it has been identified a combination and/or association or composition of EGCG with at least one of the following agents: folic acid, vitamin B12 and low or very low molecular weight hyaluronic acid (1-500 kDa), that has an inhibitory effect on tumoral cell proliferation or cells infected with HPV in vitro. Preferably the combination and/or association or composition of the invention comprises all the above actives. As demonstrated by the invention, the present combination and/or association and/or composition demonstrates a synergistic and/or additive response reducing tumoral cell proliferation or cells infected with HPV, increases the rate of apoptosis (cell death) of tumoral cells, or cells infected with HPV and induces an increased expression or production of the p53 and a decreased expression of the viral E6 and/or E7 genes, in respect to the single effect of EGCG alone or of the single substances indicated above.

### SUMMARY OF THE INVENTION

The authors have found that the administration of a combination and/or association or composition of EGCG with at least one of the following agents: folic acid, vitamin B12 and low or very low molecular weight hyaluronic acid (1-500 kDa), has an increased effect in respect to use of the substances alone in preventing and/or blocking HPV infection. Until now it was known that EGCG could be used to treat tumoral cells or cells infected by HPV, but it was not known nor suggested that these compounds, namely EGCG, folic acid, vitamin B12, and hyaluronic acid together could be utilized in the treatment of the HPV infection. The evidence of increased tumour suppressing factors provides a novel effect that can be advantageously obtained through the use of EGCG in combination and/or association with at least one of the following agents: folic acid, vitamin B12 and low or very low molecular weight hyaluronic acid (1-500 kDa).

It is therefore an object of the invention a combination comprising or consisting of the compound of formula (I)

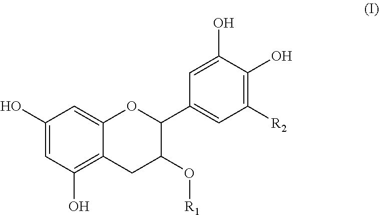

wherein R₁═H or

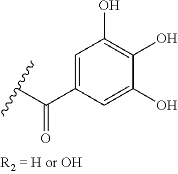 [SMILES: *C(=O)c1cc(O)c(O)c(O)c1]

or a stereoisomer or salt thereof;
and at least one further agent selected from:
   a. Folic acid or a salt or derivative thereof;
   b. Vitamin B12 or a derivative thereof;
   c. Low or very low molecular weight hyaluronic acid (1-500 kDa) or a pharmacologically acceptable salt;
for the use in prevention, control and/or inhibition of the human papillomavirus (HPV) infection.

Preferably said combination comprises:
a. the compound of formula (I) in an amount from 1 mg to 800 mg per day, preferably from 50 mg to 600 mg per day, preferably the amount is of about 200 mg per day;
b. folic acid or a salt or a derivative thereof in an amount from 1 to 400 micrograms per day, preferably from 50 to 400 micrograms per day, preferably from 200 to 400 micrograms per day, preferably about 400 micrograms per day;
and/or
c. vitamin B12 or a derivative thereof in an amount from 1 to 1000 micrograms per day, preferably from 100 to 1000 micrograms (1 mg) per day, preferably in an amount from 500 to 1000 micrograms (1 mg) per day, preferably about 1000 micrograms (1 mg) per day; and/or
d. very low/low molecular weight hyaluronic acid or a pharmaceutically acceptable salt thereof in an amount from 1 mg to 200 mg per day, preferably from 25 mg to 150 mg per day, preferably from 50 to 100 mg per day, preferably about 50 mg per day.

Preferably the combination of the invention comprises a compound of formula (I) or stereoisomer or salt thereof and at least two further agents selected from:
a. folic acid or a salt or a derivative thereof;
b. vitamin B12 or a derivative thereof;
c. very low/low molecular weight hyaluronic acid or a pharmaceutically acceptable salt thereof.

More preferably the combination of the invention comprises i) the compound of formula (I) or stereoisomer or salt thereof and ii) folic acid or a salt or a derivative thereof and iii) vitamin B12 or a derivative thereof and iv) very low/low molecular weight hyaluronic acid or a pharmaceutically acceptable salt thereof.

Preferably, said combination comprises or consists of at least the compound of formula (I) and folic acid or a salt or derivative thereof; preferably said combination comprises or consists of the compound of formula (I) and vitamin B12 or a derivative thereof; still preferably said combination comprises or consists of at least the compound of formula (I) and low or very low molecular weight hyaluronic acid (1-500 kDa) or a pharmacologically acceptable salt; more preferably said combination comprises or consists of at least the compound described in formula (I), folic acid or a salt or derivative thereof and low or very low molecular weight hyaluronic acid (1-500 kDa), or a pharmacologically acceptable salt; more preferably said combination comprises at least the compound of formula (I), vitamin B12 or a derivative thereof, and low or very low molecular weight hyaluronic acid (1-500 kDa) or a pharmacologically acceptable salt; more preferably said combination comprises or consist of at least the compound of formula (I), folic acid or a salt or derivative thereof and vitamin B12 or a derivative thereof.

Preferably, said combination comprises or consists of at least the compound of formula (I), folic acid or a salt or derivative, vitamin B12 or a derivative thereof and low or very low molecular weight hyaluronic acid (1-500 kDa) or a pharmacologically acceptable salt.

As herein used, the expression folic acid or a salt or derivative thereof refers to an agent or a mixture of agents selected from the following group: folic acid, a folic acid salt (a folate), calcium L-5-methylfolate, and (6s)-5-methyltetrahydrofolic acid preferably in the form of a glucosamine salt. Said agents are also named pteroyl-L-glutamic acid, vitamin M, vitamin B9, folacin, pteroyl-L-mono-glutamic acid and levo-methylfolate. Salts different from the calcium or the glucosamine salt are also within the meaning of folic acid salts according to the invention. Preferably, the expression folic acid or a salt or derivative is intended to mean calcium L-5-methylfolate.

As used herein, the expression vitamin B12 or a derivative thereof refers to cobalamin, cyanocobalamin, hydroxocobalamin, methylcobalamin; preferably, it is intended to mean cyanocobalamin.

As defined here, the expression low or very low molecular weight hyaluronic acid refers to hyaluronic acid with a molecular weight between 1-500 kDa; preferably, with a molecular weight between 1-100 kDa, more preferably, with a molecular weight of 1 to 20 kDa, more preferably, with a molecular weight of 3-10 kDa; a salt of low or very low molecular weight hyaluronic acid (1-500 kDa) as defined above, in a pharmacological accepted salt, comprises for example hyaluronic acid sodium salt also defined as sodium hyaluronate or sodium hyaluronate salt. Preferably, the expression low or very low molecular weight hyaluronic acid (1-500 kDa) or a pharmacologically acceptable salt is intended to mean sodium hyaluronate. Preferably, the compound of formula (I) is one of the following: (−)-epigallocatechin gallate (EGCG) and/or (−)-gallocatechin gallate (GCG) and/or (−)-epicatechin gallate (ECG) and/or (−)-catechin gallate (CG) and/or (−)-epicatechin (EC) and/or (+)-catechin (C) and/or (−)-epigallocatechin (EGC) and/or (+)-gallocatechin (GC); or combinations of the above, more preferably, the compound with the formula (I) is (−)-epigallocatechin gallate (EGCG). EGCG is a polyphenolic catechin and is the most common catechin in tea, specifically green tea, preferably, obtained from the Camellia sinensis plant.

Preferably, EGCG as it is described in the present invention is extracted or the product is obtained from part of the green tea plant, more preferably, from the Camellia sinensis plant, more preferably, from the leaves of the plant, including: (−)-epigallocatechin gallate (EGCG) and/or (−)-gallocatechin gallate (GCG) and/or (−)-epicatechin gallate (ECG) and/or (−)-catechin gallate (CG) and/or (−)-epicatechin (EC) and/or (+)-catechin (C) and/or (−)-epigallocatechin (EGC) and/or (+)-gallocatechin (GC). Furthermore, EGCG preferably, refers to epigallocatechin gallate in an enantiomerically pure form or as a racemic mixture.

Preferably, said human papillomavirus is identified through use of a HPV DNA test and a PAP test in women, whose infection shows evidence of alterations of various grades (low, intermediate, high) affecting the cells and/or benign and/or malignant lesions of the skin and/or mucous membranes in different gynaecological regions (vagina, cervix, vulva, anus, and penis). Preferably, said infection is a transitive infection or a persistent infection of HPV.

Preferably, the benign lesions of said infection are composed of common, flat, and plantar warts, genital and flat condyloma acuminata, which allow sexual transmission of the virus and arise in the areas of the penis, anus, female genitalia, urethra, perianal area, and the rectum. Preferably, the present invention supplements EGCG with at least one agent selected from the group consisting of folate, folic acid, calcium L-5-methylfolate or derivatives thereof, and/or an agent selected from group consisting of vitamin B12, cobalamin, cyanocobalamin, and/or an agent selected from the group consisting of low or very low molecular weight hyaluronic acid (1-500 kDa), or a pharmaceutically accepted salt (for example sodium hyaluronate or sodium hyaluronate salt) for use in the prevention, control and/or inhibition of the HPV infection. Preferably, the HPV infection refers to both the HPV infection in women and in men. Above all, preferably, the present invention provides EGCG, calcium L-5-methylfolate, vitamin B12 and low/very low molecular weight hyaluronic acid (1-500 kDa) for use in the prevention, control and/or inhibition of the HPV infection.

Preferably, in the combination and or association of the invention EGCG is in the amount from 1 to 800 mg/day. More preferably, EGCG is in the amount of 50 to 600 mg/day. More preferably, EGCG is in the amount of approximately 200 mg. Preferably, EGCG is administered two times a day, each administration being of approximately 100 mg.

Preferably, in the combination of the invention folic acid is administered in the amount from 1 to 400 μg/day. More preferably, folic acid is in the amount from 50 to 400 μg/day. More preferably, folic acid is in the amount from 200 to 400 μg/day, more preferably, approximately 400 μg/day. Preferably, folic acid is administered once per day. More preferably, folic acid is administered two times a day each administration of around 200 μg.

Preferably, in the combination of the invention vitamin B12 is in the amount of 1 to 1000 μg/day. More preferably, vitamin B12 is in the amount of 100 to 1000 μg (1 mg)/day, more preferably, vitamin B12 is in the amount of 500 to 1000 μg (1 mg)/day, more preferably, approximately 1000 mg (1 mg)/day. Preferably, vitamin B12 is administered once per day. More preferably, vitamin B12 is administered two times a day each administration of approximately 500 μg.

Preferably, in the combination of the invention low or very low molecular weight hyaluronic acid (1-500 kDa) is the amount of 1 mg to 200 mg/day. More preferably, low or very low molecular weight hyaluronic acid (1-500 kDa) is the amount of 50 mg to 100 mg/day. More preferably, low or very low molecular weight hyaluronic acid (1-500 kDa) is administered once per day in the amount of approximately 50 mg. More preferably, low or very low molecular weight hyaluronic acid (1-500 kDa) is administered two times a day each administration of approximately 25 mg.

In a preferred embodiment, the present invention provides EGCG in combination and/or association with at least one of the following agents: folic acid, vitamin B12, low or very low molecular weight hyaluronic acid (1-500 kDa), to be used as defined above in the prevention, control and/or inhibition of the HPV infection in both women and men. Therefore, in an embodiment, the present invention contains EGCG, folic acid, vitamin B12, low or very low molecular weight hyaluronic acid (1-500 kDa) for use in the prevention, control and/or inhibition of the HPV virus in both women and men.

Preferably, in the combination of the invention EGCG is in the form of tea leaves or of a leaf extract and/or part of the tea plant. More preferably, said leaves and/or parts of the plant are from the leaves and/or parts of the green tea plant, more preferably, Camellia Sinesis. Preferably, said leaves and/or part of the tea plant and/or said extract comprises EGCG in an amount of from about 30% by weight to about 95% by weight, preferably about 30% by weight, about 45% by weight, about 50% by weight or about 95% by weight.

In a preferred embodiment, the combination and/or association of EGCG with at least one of the following agents: folic acid, vitamin B12, and low or very low molecular weight hyaluronic acid (1-500 kDa), has a synergistic/additive effect, more effective in comparison to the use of the single substances, in the prevention and/or blocking of the HPV infection in both women and men, increasing the expression of oncosuppressive factors. Preferably, the combination or EGCG, folic acid, vitamin B12 and low or very low molecular weight hyaluronic acid (1-500 kDa) reduces the proliferation of both tumoral cells and cells infected with HPV, increases apoptosis (cell death) of both tumoral cells and cells infected with HPV, causes a greater increase in the expression or the production of p53 protein, a greater decrease in the expression of the viral E6 and/or E7 genes, in respect to the effect of EGCG alone or other agents when administered alone.

Preferably, said human papillomavirus, identified in women through use of an HPV DNA test and a PAP test, whose infection shows evidence of alterations of various grades (low, intermediate, high) affecting the cells and/or benign and/or malignant lesions of the skin and/or mucous membranes in different gynaecological regions (vagina, cervix, vulva, anus, and penis). Preferably, the benign lesions of said infection are composed of common, flat, and plantar warts, genital and flat condyloma acuminata, which allow sexual transmission of the virus and arise in the areas of the penis, anus, and female genitalia,

Preferably, the combination and/or association of EGCG with at least one the following agents, folic acid vitamin B12, and low or very low molecular weight hyaluronic acid (1-500 kDa) is administered orally simultaneously and/or via topical administration once or two times a day. It is a further object of the present invention a composition comprising or consisting of a compound of formula (I) as previously described or a stereoisomer or a salt, and at least one further agent selected between:
a. Folic acid or a salt or derivative thereof;
b. Vitamin B12 or a derivative thereof;
c. low or very low molecular weight hyaluronic acid (1-500 kDa) or a pharmaceutically acceptable salt;
   preferably, the compound of formula (I) is one of the following: (−)-epigallocatechin gallate (EGCG) and/or (−)-gallocatechin gallate (GCG) and/or (−)-epicatechin gallate (ECG) and/or (−)-catechin gallate (CG) and/or (−)-epicatechin (EC) and/or (+)-catechin (C) and/or (−)-epigallocatechin (EGC) and/or (+)-gallocatechin (GC); or combinations of the above, more preferably, the compound with the formula (I) is (−)-epigallocatechin gallate (EGCG).

Preferably, the composition of the invention comprises folic acid or a salt thereof and/or calcium L-5-methylfolate (often referred to as L-methylfolate) or another salt of (6s)-5-methyltetrahydrofolic acid such as the glucosamine salt.

Preferably, in the composition of the invention low or very low molecular weight hyaluronic acid is sodium hyaluronate with a molecular weight between 1 or 100 kDa, more preferably, between 1 and 20 kDa, more preferably, between 3 and 10 kDa.

Preferably, in the composition of the invention the vitamin B12 or a derivative thereof is cyanocobalamin.

Preferably, said composition contains at least the compound of formula (I) and folic acid or a salt or derivative; more preferably, said composition contains at least the compound of formula (I) and vitamin B12 or a derivative thereof; more preferably, said composition contains at least the compound with formula (I) and low or very low molecular weight hyaluronic acid (1-500 kDa) or a pharmaceutically acceptable salt.

Preferably, said composition comprises or consists of at least the compound with formula (I), folic acid or a salt or derivative and vitamin B12 or a derivative; more preferably, said composition comprises or consists of at least the compound of formula (I), folic acid and low or very low molecular weight hyaluronic acid (1-500 kDa) or a pharmaceutically acceptable salt; more preferably, said composition comprises or consists of at least the compound of formula (I), vitamin B12 or its derivative and low or very low molecular weight hyaluronic acid (1-500 kDa) or a pharmaceutically acceptable salt.

In a preferred embodiment the composition of the invention comprises or consists of: i) the compound of formula (I) or stereoisomer or salt thereof and ii) folic acid or a salt or a derivative thereof and iii) vitamin B12 or a derivative thereof and iv) very low/low molecular weight hyaluronic acid or a pharmaceutically acceptable salt thereof.

Preferably the composition of the invention comprises:
a. the compound of formula (I) in an amount from 1 mg to 800 mg, preferably from 50 mg to 600 mg, preferably about 200 mg, preferably about 100 mg;
b. folic acid or a salt or a derivative thereof in an amount from 1 to 400 micrograms, preferably from 50 to 400 micrograms, preferably from 200 to 400 micrograms, preferably about 400 micrograms, preferably about 200 micrograms; and/or
c. vitamin B12 or a derivative thereof in an amount from 1 to 1000 micrograms, preferably from 100 to 1000 micrograms (1 mg), preferably in an amount from 500 to 1000 micrograms (1 mg), preferably about 1000 micrograms (1 mg), preferably about 500 micrograms; and/or
d. very low/low molecular weight hyaluronic acid or a pharmaceutically acceptable salt thereof in an amount from 1 mg to 200 mg, preferably from 25 mg to 150 mg, preferably from 50 to 100 mg, preferably about 50 mg, preferably about 25 mg.

Preferably, said composition is used in the prevention, control and/or inhibition of the HPV infection, in a transient or a persistent state, preferably, said human papillomavirus, identified through use of a HPV DNA test and PAP test in women, whose infection shows evidence of alterations of various grades (low, intermediate, high) affecting the cells and/or benign and/or malignant lesions of the skin and/or mucous membranes in different gynaecological regions (vagina, cervix, vulva, anus, and penis). Preferably, the benign lesions of said infection are composed of common, flat, and plantar warts, genital and flat condyloma acuminata, which allow sexual transmission of the virus and arise in the areas of the penis, anus, and female genitalia.

Still preferably, in the composition EGCG is in the form of tea plant leaves or tea plant leaf extract, preferably said tea plant leaves are green tea plant leaves, preferably Camellia sinensis, more preferably said tea plant leaves comprise EGCG in an amount of 30% by weight to 95% by weight, even more preferably about 30% by weight, 45% by weight, 50% by weight or 95% by weight.

Preferably the composition comprises EGCG in the amount from 1 to 800 mg/day. More preferably, EGCG is in the amount of 50 to 600 mg. More preferably, EGCG is in the amount of approximately 200 mg. More preferably, EGCG in the composition is administered once a day; still preferably EGCG in the composition is administered two times a day, each administration of approximately 100 mg.

Preferably, the composition comprises folic acid or a salt or derivative thereof in the amount from 1 to 400 μg. More preferably, folic acid is in the amount from 50 to 400 μg. More preferably, folic acid is in the amount from 200 to 400 μg, more preferably is in the amount of about 400 μg, even more preferably folic acid is in the amount of about 200 μg. Preferably, folic acid in the composition is administered once per day. More preferably, folic acid in the composition is administered two times a day each administration being of about 200 μg. Preferably, in the composition vitamin B12 is in the amount of 1 to 1000 μg. More preferably, vitamin B12 is in the amount of 100 to 1000 μg (1 mg), more preferably, vitamin B12 is in the amount of 500 to 1000 μg (1 mg), more preferably of about 1000 μg (1 mg) even more preferably in the amount of 500 μg. Preferably, vitamin B12 in the composition is administered once per day. More preferably, vitamin B12 is administered two times a day each administration of approximately 500 μg.

Preferably, the composition contains low or very low molecular weight hyaluronic acid (1-500 kDa) in the amount of 1 mg to 200 mg. More preferably, low or very low molecular weight hyaluronic acid (1-500 kDa) is in the amount of 50 mg to 100 mg, preferably about 50 mg, preferably about 25 mg. More preferably, low or very low molecular weight hyaluronic acid (1-500 kDa) as contained in the composition of the invention is administered once per day in the amount of approximately 50 mg. More preferably, low or very low molecular weight hyaluronic acid (1-500 kDa) is administered two times a day each administration of approximately 25 mg.

Preferably, said composition is administered orally and/or via topical administration. Preferably, said composition is administered once or two times a day.

Preferably, said composition is in the form of a tablet, a hard capsule, a soft gel capsule, a powder, a syrup, a drinking liquid, a gel, an ointment, a cream.

Optionally, said composition is used in combination with further therapeutic invention. Preferably, said therapeutic invention is selected from the following: surgery, with the aim of removing lesions which develop as a result of an infection from any variant of the human papillomavirus.

It is a further object of the present invention a composition comprising or consisting of: a compound of formula (I) as previously defined and preferably, selected from the following: (−)-epigallocatechin gallate (EGCG) and/or (−)-gallocatechin gallate (GCG) and/or (−)-epicatechin gallate (ECG) and/or (−)-catechin gallate (CG) and/or (−)-epicatechin (EC) and/or (+)-catechin (C) and/or (−)-epigallocatechin (EGC) and/or (+)-gallocatechin (GC), or mixtures thereof; more preferably, EGCG refers to gallocatechin gallate; and at least one, preferably at least two of the following agents:
folic acid or salt or a derivative; preferably, calcium L-5-methylfolate or another salt of (6s)-5-methyltetrahydrofolic acid such as the glucosamine salt;
an agent selected from the following: cobalamin or cyanocobalamin or methylcobalamin or hydroxocobalamin; preferably, cyanocobalamin;
an agent selected from the following: low or very low molecular weight hyaluronic acid (1-500 kDa), and a pharmacologically acceptable salt (for example sodium hyaluronate or sodium hyaluronate salt); preferably, low or very low molecular weight hyaluronic acid and sodium hyaluronate or sodium hyaluronate salt has a molecular weight between 1-500 kDa; more preferably, has a molecular weight between 1-100 kDa, more preferably, a molecular weight between 1 to 20 kDa, more preferably, a molecular weight between 3-10 kDa;
and a pharmaceutically acceptable excipient or diluent.

Preferably, said excipient and/or diluent is selected from the following: calcium phosphate, dicalcium phosphate, microcrystalline cellulose, magnesium stearate (or generally magnesium salts of fatty acids), silicon dioxide, sucrose, gum arabic, corn starch, medium-chain triglycerides, tricalcium phosphate, cross-linked sodium carboxymethylcellulose, hydroxypropyl methylcellulose, polyethylene glycol, titanium dioxide, polyvinylpyrrolidone, talcum powder, erythritol, xylitol, steviol glycosides, and sucralose.

Preferably, the composition is used in the prevention, control and/or inhibition or the HPV infection, preferably, said human papillomavirus, identified in women through use of an HPV DNA test and a PAP test, whose infection shows evidence of alterations of various grades (low, intermediate, high) affecting the cells and/or benign and/or malignant lesions of the skin and/or mucous membranes in different gynaecological regions (vagina, cervix, vulva, anus, and penis). Preferably, the benign lesions of said infection are composed of common, flat and plantar warts, genital and flat condyloma acuminata, which allow sexual transmission of the virus and arise in the areas of the penis, anus, and female genitalia.

Still preferably, in the above identified composition, EGCG is comprised in powdered leaves of a tea plant or in an extract thereof, preferably is in the form of tea leaves or tea-leaf extract, more preferably, said tea leaves are green tea leaves, more preferably, said tea leaves contains said agents in an amount of from 30% by weight to 95% by weight, preferably about 30% by weight, about 45% by weight, about 50% by weight or about 95% by weight.

Preferably, EGCG is in the amount from 1 to 800 mg. More preferably, EGCG in the amount of 50 to 600 mg. More preferably, EGCG is in the amount of approximately 200 mg. Even more preferably, EGCG is in the amount of approximately 100 mg. More preferably, in the composition of the invention EGCG is administered two times a day, each administration being of approximately 100 mg.

Preferably, the composition comprises folic acid in the amount from 1 to 400 μg. More preferably, folic acid is in the amount from 50 to 400 μg. More preferably, folic acid is in the amount from 200 to 400 μg, more preferably approximately 400 μg, even more preferably 200 μg. Preferably, folic acid in the composition is administered once per day. More preferably, in the composition of the invention folic acid is administered two times a day, each administration being of about 200 μg.

Preferably, the composition comprises vitamin B12 in the amount of 1 to 1000 μg. More preferably, vitamin B12 is in the amount of 100 to 1000 μg (1 mg), more preferably, vitamin B12 is in the amount of 500 to 1000 μg (1 mg), more preferably approximately 1000 μg (1 mg), even more preferably vitamin B12 is in the amount of about 500 μg. Preferably, in the composition of the invention vitamin B12 is administered once per day. More preferably, vitamin B12 is administered two times a day each administration of approximately 500 μg. Preferably, the composition comprises low or very low molecular weight hyaluronic acid (1-500 kDa) is the amount of 1 mg to 200 mg. More preferably, low or very low molecular weight hyaluronic acid (1-500 kDa) is the amount of 25 mg to 100 mg, preferably in the amount of about 50 mg, more preferably in the amount of about 25 mg. More preferably, in the composition of the invention low or very low molecular weight hyaluronic acid (1-500 kDa) is administered once per day in the amount of approximately 50 mg. More preferably, low or very low molecular weight hyaluronic acid (1-500 kDa) is administered two times a day each administration of approximately 25 mg.

Preferably, said composition is administered orally and/or via topical administration. Preferably, said composition is administered once or two times a day.

Preferably, said composition is in the form of a tablet, a hard capsule, a soft gel capsule, a powder, a syrup, a drinking liquid, a gel, an ointment, a cream.

Optionally, said composition is used in combination with further therapeutic invention. Preferably, said therapeutic invention is selected from the following: surgery, with the aim of removing lesions which develop as a result of an infection from any variant of the human papillomavirus.

It is a further objective of the present invention to have a pharmaceutical composition, or a dermocosmetic, or a medical device or a supplement or a dietary product, preferably, a food for special medical purposes, or a drinkable product, or a topical formulation containing the compound of formula (I) as previously defined, and at least one of the following ingredients:
folic acid or salt or a derivative; preferably, calcium L-5-methylfolate or another salt of (6s)-5-methyltetrahydrofolic acid such as the glucosamine salt;
an agent selected from the following: cobalamin or cyanocobalamin or methylcobalamin or hydroxocobalamin; preferably, cyanocobalamin;
an agent selected from the following; and low or very low molecular weight hyaluronic acid (1-500 kDa), and a pharmacologically acceptable salt (for example sodium hyaluronate or sodium hyaluronate salt); preferably, low or very low molecular weight hyaluronic acid and sodium hyaluronate or sodium hyaluronate salt has a molecular weight between 1-500 kDa; more preferably, with a molecular weight between 1-100 kDa, more preferably, with a molecular weight of 1 to 20 kDa, more preferably, with a molecular weight of 3-10 kDa;
a pharmaceutically acceptable excipient or diluent.

Preferably, the compound of formula (I) is selected from: epigallocatechin gallate (EGCG), catechin, gallocatechin and epicatechin gallate (ECG). More preferably, in the composition, said agent is from the group consisting of: EGCG, epigallocatechin-3-gallate, (−)-epigallocatechin-3-gallate, (−)-gallocatechin-3-O-gallate (GCG), (−)-gallocatechin-3-gallate, (+)-catechin (C), (−)-epicatechin (EC), (+)-gallocatechin (GC), (−)-epigallocatechin, (−)-epicatechin-3-gallate, (−)-catechin-3-gallate, and (+)-catechin-3-gallate. More preferably, said agent is epigallocatechin gallate (EGCG) under the form of green-tea extract.

Preferably, said pharmaceutical composition, or dermocosmetic, or medical device, or supplement, a dietary product, preferably, food for medical purposes, or drinkable product, or topical formulation for use in the prevention, control and/or inhibition of the HPV infection, identified through use of a HPV DNA test and a PAP test in women, whose infection shows evidence of alterations of various grades (low, intermediate, high) affecting the cells and/or benign and/or malignant lesions of the skin and/or mucous membranes in different gynaecological regions (vagina, cervix, vulva, anus, and penis). Preferably, said infection is a transitive infection or a persistent infection of HPV. Preferably, the benign lesions of said infection are composed of common, flat, and plantar warts, genital and flat condyloma acuminata, which allow sexual transmission of the virus and arise in the areas of the penis, anus, female genitalia, urethra, perianal area, and the rectum.

Preferably, in said pharmaceutical composition, or dermocosmetic, or medical device, or supplement, a dietary product, preferably, food for medical purposes, or drinkable product, or topical formulation, EGCG is in the form of tea leaves or of a leaf extract and/or part of the tea plant. Preferably, said leaves and/or parts of the plant are from the leaves and/or parts of the green tea plant, more preferably, Camellia Sinesis. Preferably, said leaves and/or part of the tea plant and/or said extract contains EGCG in a quantity between 30% to 95% by weight, more preferably, approximately 30%, 45%, 50% or 95% by weight.

Preferably, said pharmaceutical composition, or dermocosmetic, or medical device, or supplement, a dietary product, preferably, food for medical purposes, or drinkable product, or topical formulation contains EGCG in the amount from 1 to 800 mg. More preferably, EGCG in the amount of 50 to 600 mg. More preferably, EGCG is in the amount of approximately 200 mg, even more preferably in the amount of about 100 mg. More preferably, EGCG is administered two times a day of each administration of approximately 100 mg.

Preferably, said pharmaceutical composition, or dermocosmetic, or medical device, or supplement, a dietary product, preferably, food for medical purposes, or drinkable product, or topical formulation contains folic acid in the amount from 1 to 400 μg. More preferably, folic acid is in the amount from 50 to 400 μg. More preferably, folic acid is in the amount from 200 to 400 μg, more preferably, approximately 400 μg. Preferably, folic acid is administered once per day. More preferably, folic acid is administered two times a day each administration of around 200 μg.

Preferably, said pharmaceutical composition, or dermocosmetic, or medical device, or supplement, a dietary product, preferably, food for medical purposes, or drinkable product, or topical formulation contains vitamin B12 is in the amount of 1 to 1000 μg. More preferably, vitamin B12 is in the amount of 100 to 1000 μg (1 mg), more preferably, vitamin B12 is in the amount of 500 to 1000 μg (1 mg), more preferably approximately 1000 μg (1 mg), even more preferably about 500 g. Preferably, vitamin B12 is administered once per day. More preferably, vitamin B12 is administered two times a day each administration of approximately 500 μg.

Preferably, said pharmaceutical composition, or dermocosmetic, or medical device, or supplement, a dietary product, preferably, food for medical purposes, or drinkable product, or topical formulation contains low or very low molecular weight hyaluronic acid (1-500 kDa) in the amount of 1 mg to 200 mg. More preferably, low or very low molecular weight hyaluronic acid (1-500 kDa) is the amount of 25 mg to 100 mg, preferably 50 mg, preferably 25 mg. More preferably, low or very low molecular weight hyaluronic acid (1-500 kDa) is administered once per day in the amount of approximately 50 mg. More preferably, low or very low molecular weight hyaluronic acid (1-500 kDa) is administered two times a day each administration of approximately 25 mg.

Preferably, said excipient and/or diluent is selected from the following: calcium phosphate, dicalcium phosphate, microcrystalline cellulose, magnesium stearate (or generally magnesium salts of fatty acids), silicon dioxide, sucrose, gum arabic, corn starch, medium-chain triglycerides, tricalcium phosphate, cross-linked sodium carboxymethylcellulose, hydroxypropyl methylcellulose, polyethylene glycol, titanium dioxide, polyvinylpyrrolidone, talcum powder, erythritol, xylitol, steviol glycosides, and sucralose.

Preferably, said pharmaceutical composition, supplement or food for medical purposes, or a drinkable product is administered orally. Preferably, said formulation is administered topically. Preferably, said supplement, or food for medical purposes or drinkable product, or medical device for topical use is administered once or twice a day. Preferably, said pharmaceutical composition, supplement, food for medical purposes, or a drinkable product, or formulation for topical use is in the form of a tablet, a hard capsule, a soft gel capsule, a powder, a syrup, a drinking liquid, a dietary supplement, an edible bar or an edible snack, a gel, a cream, an ointment.

Specifically, said supplement could be any type of dietary supplement. Furthermore, said dietary supplement preferably, includes a food for special medical purposes. A food for special medical purposes is a food that is prepared or formulated with the intention of dietary management of patients, including infants, to be used under medical supervision; it is designed for the complete or partial feeding of patients with a limited capacity, or impaired ability to take, digest, absorb, or excrete common foods or nutrients or metabolites contained within, or with other nutritional needs determined by clinical conditions or cannot be accomplished solely with modification of the normal diet.

Optionally, said supplement or food for special medical purposes or drinkable product or topical formulation is for use in combination with a therapeutic intervention, said therapeutic invention is selected from the following: surgery, with the aim of removing lesions which develop as a result of an infection from any variant of the human papillomavirus.

A further object of the invention is the non-therapeutic use of said composition as previously defined, or of said pharmaceutical composition, or dermocosmetic, or medical device, or a supplement, or dietary product, preferably, a food for medical purposes, or a drinkable product, or a topical formulation in the nutraceutical sector or as a base ingredient in the preparation of supplements and/or dietary products and/or food for special medical purposes. Said supplements and/or dietary products and/or food for special medical purposes are non-therapeutic products useful in the prevention, control and/or inhibition of the human papillomavirus (HPV) preferably, said human papilloma virus whose infection shows evidence of alterations of various grades (low, intermediate, high) affecting the cells and/or benign and/or malignant lesions of the skin and/or mucous membranes in different gynaecological regions (vagina, cervix, vulva, anus, and penis). Preferably, the benign lesions of said infection are composed of common, flat and plantar warts, genital and flat condyloma acuminata, which allow sexual transmission of the virus and arise in the areas of the penis, anus, and female genitalia.

Another object of the invention is the combination or the composition, or pharmaceutical composition, or dermocosmetic, or medical device, or supplement, or dietary product, preferably, a food for medical purposes, or drinkable product, or topical formulation each comprising the compound of formula (I) and at least one other agent, preferably at least two further agents selected from: a) folic acid or a salt or derivative, b) vitamin B12 or a derivative, c) low or very low molecular weight hyaluronic acid or a pharmaceutically acceptable salt; in which the compound with formula (I) and the other agent or other agents reduce the proliferation of tumoral cells or cells infected with HPV, increase apoptosis (cell death) of the tumoral cells or cells infected with HPV, show a major increase in the expression or the production of the proteins p53 and/or a major decrease in the expression of the viral proteins E6 and/or E7 in respect to singular agents.

The present invention will be illustrated by means of non-limiting examples in reference to the following figures.

### BRIEF DESCRIPTION OF THE DRAWINGS

FIG. 1. Percentage of apoptosis in Hela cells after treatment with EGCG, Fol, B12 and Hyal. ANOVA Bonferroni post-test; *p<0.05, ***p<0.001, ****p<0.0001 vs ctrl; ^{#####}p<0.0001 vs EGCG; ^{§§§§} p<0.0001 vs EGCG+Fol+B12+Hyal.

FIG. 2. P53 expression in Hela cells after treatment with EGCG, Fol, B12 and Hyal. ANOVA Bonferroni post-test was performed; *p<0.05, ****p<0.0001 vs ctrl; ^{####}p<0.0001 vs EGCG; ^{@@}p<0.01, ^{@@@}p<0.001 vs EGCG+Fol+B12+Hyal; ^{$$}p<0.001, ^{§§§§} p<0.0001 vs EGCG+Fol+B12+Hyal.

FIG. 3. E6/E7 expression in Hela cells after EGCG, Fol, B12 and Hyal treatment. ANOVA Bonferroni post-test was performed; *p<0.05, **p<0.01, ***p<0.001 vs ctrl.

FIG. 4. Rb expression in Hela cells after EGCG, Fol, B12 and Hyal treatment.

### DETAILED DESCRIPTION OF THE INVENTION

The following describes definitions of various terms and expressions used in the application to describe the invention. It should be understood that the definitions of terms and expressions used in one aspect or form of the invention apply equally to any aspect or form of invention described herein in which those terms and expressions appear.

This is also true regardless of where, i.e., in which section, within this application, these terms are defined or discussed.

Furthermore, also if the present application mentions an embodiment separately, it is understood that any embodiment and its characteristics may be freely combined with any other embodiment and their respective characteristics, also in absence of its explicit mention within the text. Such combinations of an embodiment with another, or one or more characteristics of an embodiment with one or more characteristics of another embodiment, are, therefore, part of the description of the present application as filed, as understood by the skilled person.

In this application the use of the singular (for example “a” and “the”) can include the plural, unless otherwise specified. Additionally, the use of the term “which include” and other grammatical forms such as “which includes” is not limiting. In the present context, the term including has a broad meaning and can be used to mean “which include,” “which cover” “which contains.”

Includes the element or elements explicitly stated and allows, but does not require, the presence of another element or elements non explicitly stated. In addition to its broader meaning, in this context the term “comprising” includes also the limited meaning of “consisting of” according to which only the explicitly stated element or elements are present and none others. Furthermore, the term “comprising” also includes “essentially consisting of” which means that one or more elements may be present in addition to those explicitly stated, provided that the additional element or elements do not alter the technical effect obtained from the explicitly stated element or elements.

In the present context, the term “approximately” or the term “about”, which are herein used interchangeably, when it refers to a particular value, for the example an endpoint or the endpoints of a time interval, includes and describes, other than the value specifically state, a degree of variation around the specified value. Such variation can, for example, derive from the normal variability in measurement, for example, in the weighing or apportioning of various substances with methods known to someone experienced in the art. The term “approximately” shall be understood to include and describe a range of variability above or below the specified value, the described percentage values being relative to the same specified value as follows. The term “approximately” may be understood to include and describe a variability of ±5.0%. The term “approximately” may be understood to include and describe a variability of ±4.9%. The term “approximately” may be understood to include and describe a variability of ±4.8%. The term “approximately” may be understood to include and describe a variability of ±4.7%. The term “approximately” may be understood to include and describe a variability of ±4.6%. The term “approximately” may be understood to include and describe a variability of ±4.5%. The term “approximately” may be understood to include and describe a variability of ±4.4%. The term “approximately” may be understood to include and describe a variability of ±4.3%. The term “approximately” may be understood to include and describe a variability of ±4.2%. The term “approximately” may be understood to include and describe a variability of ±4.1%. The term “approximately” may be understood to include and describe a variability of ±4.0%. The term “approximately” may be understood to include and describe a variability of ±3.9%. The term “approximately” may be understood to include and describe a variability of ±3.8%. The term “approximately” may be understood to include and describe a variability of ±3.7%. The term “approximately” may be understood to include and describe a variability of ±3.6%. The term “approximately” may be understood to include and describe a variability of ±3.5%. The term “approximately” may be understood to include and describe a variability of ±3.4%. The term “approximately” may be understood to include and describe a variability of ±3.3%. The term “approximately” may be understood to include and describe a variability of ±3.2%. The term “approximately” may be understood to include and describe a variability of ±3.1%. The term “approximately” may be understood to include and describe a variability of ±3.0%. The term “approximately” may be understood to include and describe a variability of ±2.9%. The term “approximately” may be understood to include and describe a variability of ±2.8%. The term “approximately” may be understood to include and describe a variability of ±2.7%. The term “approximately” may be understood to include and describe a variability of ±2.6%. The term “approximately” may be understood to include and describe a variability of ±2.5%. The term “approximately” may be understood to include and describe a variability of ±2.4%. The term “approximately” may be understood to include and describe a variability of ±2.3%. The term “approximately” may be understood to include and describe a variability of ±2.2%. The term “approximately” may be understood to include and describe a variability of ±2.1%. The term “approximately” may be understood to include and describe a variability of ±2.0%. The term “approximately” may be understood to include and describe a variability of ±1.9%. The term “approximately” may be understood to include and describe a variability of ±1.8%. The term “approximately” may be understood to include and describe a variability of ±1.7%. The term “approximately” may be understood to include and describe a variability of ±1.6%. The term “approximately” may be understood to include and describe a variability of ±1.5%. The term “approximately” may be understood to include and describe a variability of ±1.4%. The term “approximately” may be understood to include and describe a variability of ±1.3%. The term “approximately” may be understood to include and describe a variability of ±1.2%. The term “approximately” may be understood to include and describe a variability of ±1.1%. The term “approximately” may be understood to include and describe a variability of ±1.0%. The term “approximately” may be understood to include and describe a variability of ±0.9%. The term “approximately” may be understood to include and describe a variability of ±0.8%. The term “approximately” may be understood to include and describe a variability of ±0.7%. The term “approximately” may be understood to include and describe a variability of ±0.6%. The term “approximately” may be understood to include and describe a variability of ±0.5%. The term “approximately” may be understood to include and describe a variability of ±0.4%. The term “approximately” may be understood to include and describe a variability of ±0.3%. The term “approximately” may be understood to include and describe a variability of ±0.2%. The term “approximately” may be understood to include and describe a variability of ±0.1%. The term “approximately” in reference to a particular value, may be understood include and describe the cited value itself regardless of any explicit mention of that value in which it is included; also in the absence of the explicit indication that the term “approximately” includes the cited value, this particular cited value is nevertheless included in the range of variability created from the term “approximately”, and is thus described.

As stated above, the present invention refers to epigallocatechin gallate (EGCG) in combination and/or association with at least one of the following agents: folic acid, vitamin B12, low or very low molecular weight hyaluronic acid (1-500 kDa), to be used as defined above in the prevention, control and/or inhibition of the HPV infection in both women and men. In a correlated aspect, the present invention regards a method of prevention, control and/or inhibition of the HPV infection in both women and men in need or potentially in need, comprising of the administration of EGCG, folic acid, vitamin B12 and low or very low molecular weight hyaluronic acid (1-500 kDa) to said subject.

Another correlated aspect, the present invention regards the use of EGCG, folic acid, vitamin B12 and low/or very low weight hyaluronic acid (1-500 kDa) in production of a product for the prevention, control and/or inhibition of the HPV infection in a subject (both in women and in men).

In the present context, the term subject refers to a mammal, preferably, a human. The subject is of need, potential need or suspected need of the prevention, control and/or inhibition of the HPV infection. The subject has need of treatment for the HPV infection.

In these cases, this need, typically, is determined upon previous diagnosis of the HPV infection.

In such cases, in which there is already a diagnosis or diagnoses of a pathological condition or pathological conditions, the invention as described herein may be used for the treatment of existing pathologies (for example HPV infection which is curable), prevention at the same time of another infection (for example the persistence of the infection; appearance of signs such as common, flat and plantar warts, genital and flat condylomata acuminata; neoplastic disease). However, it is anticipated that the subject matter of the invention as it is described here could apply to subjects that have suspected need. Such subjects may be at risk of developing this condition (for example sexually active populations without a stable partner, immune-depressed subjects, subjects who use oral contraceptives.)

Consequently, in the present context, the term “treat” or the grammatically correct variations such as “treatment,” “that treats” etc. refer to the improvement, also temporary improvement, which includes, but does not require, the complete abolition of the pathological state. In the broadest sense, the treatment of the HPV infection refers to, in this context, the observation of at least a regression, at least a partial regression, of the infection. Specifically, the presented treatment includes the restoration of the correct architecture and function of the affected epithelium and/or mucous membranes and regression of the lesions associated with the HPV infection.

In the present context, the term infection refers to the processes that causes the entrance and the multiplication of a microorganism, both of a virus or a bacterium, in the tissues of a host. The infectious disease is the manifestation of the infection.

In the present context, the term “infection of the human papillomavirus” refers to a condition in which, following the penetration and successful replication and/or integration of the virus into the cells and the genome of the host, the development of benign lesions, that comprises of composed of common, flat and plantar warts, genital and flat condyloma acuminata and/or lesions of various grades (low, intermediate, high), in the cells of various tissues of different gynaecological and urological area (vagina, cervix, vulva, anus, penis).

An HPV infection may be diagnosed with the through use of an HPV DNA test and PAP test in women. In general, the treatment of the HPV infection may include surgery of various types such as scalpel treatments, laser, cryotherapy, diathermocoagulation, with the aim of removing lesions resulting from the infection of any strain of HPV.

Any of such treatments of the HPV infection may be used in combination with EGCG, folic acid, vitamin B12 and low or very low molecular weight hyaluronic acid (1-500 kDa) according to the present invention.

In the present context, the term “prevent” or grammatically correct variants such as “prevention” refer to scenarios in which the use of the methods described here are applied to avert the possibility, suspected, or expected occurrence of the HPV infection. These suspected cases arise from the fact that the infection may derive (also in the absence of a corresponding diagnosis) from the history of the subject, for example in the case in which the subject has had a sexual interaction with an HPV positive subject. In this scenario, one may suspect that the subject is positive for the HPV infection. Such suspicion that an HPV infection may occur (also in the absence of a corresponding diagnosis) may be applied also towards subjects at risk of developing this condition, for example sexually active subjects that do not have a stable partner, immune-depressed subjects, subjects who use oral contraceptives.

In the present context, the term “EGCG” includes and describes a substance with the chemical formula C₂₂H₁₈O₁₁ i.e., epigallocatechin gallate (EGCG), (−) epigallocatechin-3-gallate (EGCG), (−)-gallocatechin-3-O-gallate (GCG). In the present context, the term “epigallocatechin gallate” includes and describes a substance with the chemical formula: C₂₂H₁₈O₁₁, i.e. (−)-epicatechin-3-gallate (ECG), (−)-catechin-3-gallate (CG) and (+)-catechin-3-gallate (CG). In the present context the term “catechin” includes and describes the substances with the chemical formula C₁₅H₁₄O₆ i.e., catechin, (+)-catachin (C), (−)-epicatechin (EC). In the present context, the term “gallotechin” includes and describes a substance with the chemical formula: C₁₅H₁₄O₇, i.e., gallotechin, (+)-gallotechin (GC), (−)-epigallocatechin (EGC).

EGCG is contained in various foods, each of which, or any combination of which, may be used in the present invention as a source of EGCG. Specifically, in the present invention, the source of EGCG may be, green tea, white tea, oolong tea, black tea, carob flour, blackberries, apples, raspberries, pecans, hazelnuts, blueberries, prunes, peaches, avocados, strawberries and/or onions. The carob flour may be obtained from the fruit of the Ceratonia plant.

Preferably, the source of EGCG is tea, the preferred sources for EGCG include tea, specifically: green tea, white tea, oolong tea and black tea. As a source of EGCG in the present invention any source of tea may be used. More preferably, Camellia sinensis is used. As a source of EGCG in the present invention, any part of the tea plant, or the entirety of the tea plant may be used. More preferably, the leaves of the tea plant are used. More preferably, in the present invention the leaves of Camellia sinensis is used. Tea leaves can contain different quantities of EGCG, specifically from 30% to 95% by weight. For example, 30% by weight, 45% by weight, 50% by weight and 95% by weight. Such quantities may be determined by titration and depend on the purity and the level of processing of the raw material. More preferably, EGCG is used in the present invention as a dry tea extract, more preferably, a dry green tea extract.

In the present context, the expression “folic acid or a salt or derivative” includes and describes the substances with the chemical formula C₁₉H₁₉N₇O₆ (folic acid, folate, vitamin B9, pteroyl-L-glutamic acid, vitamin M, folacin, pteroyl-L-mono-glutamic acid), C₂₀H₂₃CaN₇O₆ (L-calcium methylfolate, L-5-methyltetrahydrofolate, levo-methylfolate). Also included are folic acid salts and additional salts of L-5-methyltetrahydrofolic acid such as sodium, potassium, magnesium, and organic salts such as glucosamine, galactosamine, methylamine. Preferably, the expression “folic acid or a salt or derivative” refers to L-calcium methylfolate (CAS No. 151533-22-1), also commonly known as calcium methylfolate, L-5-calcium methylfolate, L-5-methyltetrahydrofolate, levo-methylfolate.

In the present context, the expression “vitamin B12 or a derivative” includes and describes the substances with the chemical formula C₆₃H₈₈CoN₁₄O₁₄P (vitamin B12, cobalamin, cyanocobalamin). C₆₂H₈₉CoN₁₃O₁₅P (hydroxocobalamin), C₆₃H₉₁CoN₁₃O₁₄P (methylcobalamin). Preferably, vitamin B12 refers to cyanocobalamin.

In the present context, the expression “low or very low molecular weight hyaluronic acid or a pharmacologically acceptable salt” includes and describes the substances with the chemical formula (C₁₄H₂₁NO₁₁)ₙ (hyaluronic acid, hyaluronan), (C₁₄H₂₀NO₁₁Na)ₙ (hyaluronic acid sodium salt, sodium hyaluronate, sodium hyaluronate salt. For “low or very low weight” is intended hyaluronic acid, as defined above, with a molecular weight between 1 and 500 kDa, preferably, in the range of 1 to 100 kDa or 1 to 20 kDa or 3 to 10 kDa. Preferably, hyaluronic acid according to the present invention is sodium hyaluronic.

An embodiment of the present invention includes the combination and/or association of EGCG with at least one of the following agents: folic acid, vitamin B12, low or very low molecular weight hyaluronic acid (1-500 kDa).

An embodiment of the present invention includes the combination and/or association of EGCG, with folic acid, vitamin B12, low or very low molecular weight hyaluronic acid (1-500 kDa).

An embodiment of the present invention refers to the following respective amounts:
Green tea extract 444.5 mg (Tit. 45% epigallocatechin gallate (EGCG) 200 mg)
Hyaluronic acid 50 mg
Vitamin B12 1000 μg (1 mg)
Folic acid 400 μg

The agents of the present invention may exist or be used according to the invention in any form of salt, solvate, stereoisomer, zwitterion and/or isotope.

The salts of the agents in the present invention are, preferably, pharmaceutically acceptable. Pharmaceutically acceptable salts include non-toxic conventional salts obtained by salification of an agent in the present invention with inorganic acids (for example hydrochloric, hydrobromic, sulphuric or phosphoric acids) or with organic acids (for example acetic, propanoic, succinic, benzoic, sulfanilic, 2-acetoxy-benzoic, cinnamic, mandelic, salicylic, glycolic, lactic, oxalic, malic, maleic, malonic, fumaric, tartaric, citric, p-toluenesulfonic, methanesulfonic, ethanosulfonic, or naphthalenesulfonic acids). For a review on pharmaceutically accepted acids please see Berge S. M. et al., J. Pharm. Sci. 1977, 66, 1-19. Furthermore, additional salts with a pharmaceutically accepted base can be formed using inorganic or organic bases such as triethylamine, ethanolamine, triethanolamine, dicyclohexylamine, ammonium hydroxide, pyridine, glucosamine, galactosamine. The term “inorganic base” in the present context, has its ordinary meaning to someone with ordinary competence in the field and generally refers to an inorganic compound that can act as a proton accepter. Also, the term “organic base” in the present context, has its ordinary meaning to someone with ordinary competence in the field and generally refers to an organic compound that can act as a proton acceptor. Others pharmaceutically acceptable salts include pharmaceutically acceptable salts of alkaline metals or alkaline earth-metals such as sodium, potassium, calcium, and magnesium. The invention relates to the use of all possible stoichiometric and non-stoichiometric forms of the salts of the agents of the present invention. Furthermore, the agents in the present invention may exist in both solvate and non-solvate with pharmaceutically acceptable solvents such as water, ethanol, and other similar examples. Some agents of the present invention, specifically, EGCG, folic acid and hyaluronic acid may exist in different stereoisomeric or polymeric forms (for example, they may contain one or more asymmetrical carbon atoms, or present as molecules with different molecular weights). In the treatment of the HPV infection according to the present invention they may be all stereoisomers of the agents in the present invention may be used, specifically EGCG and folic acid. Specifically, the compound with formula (I) in which R₁ and R₂ have the given previous meanings, possesses the chiral centres indicated with the asterisks.

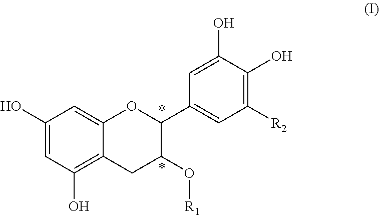

Each of these chiral centres may exist in the R or S form, therefore, the compound with formula (I) includes the stereoisomers (R,R), (R,S), (S,R), (S,S) and their mixtures. The use of single stereoisomers (enantiomers and diastereomers) and their mixtures are included in the scope of this present invention. The racemic mixes may be separated to obtain a single enantiomer using preparative HPLC with a column with a chiral stationary phase or with a column designed for the production of single enantiomers using methods known to experts in the field. Different forms of polymeric hyaluronic acid may be used, with a molecular weight in the range of 1-500 kDa, preferably, between 1-100 kDa or 1-20 kDa, or 3-10 kDa.

The agents of the present invention may exist in the form of zwitterions, all of which may be used in the prevention, control and/or inhibition of the HPV infection according to the present invention. Similarly, it is understood that the agent in the present invention may exists in tautomeric forms and also their use is covered in the scope of the present invention.

The invention includes the use of all of the suitable isotopic variations of the agents of the present invention. An “isotopic variation” of agent of an agent of the present invention is defined as an agent in which one atom is substituted with another atom which has the same atomic number but a different atomic mass than the atom mass usually found in nature. Example isotopes that may be incorporated into agents of the invention include 2H, 3H, 13C, 14C, 15N, 170, 180, 31P, 32p, 355, 18F, 36Cl. Some isotopic variations of the invention, for example those in which as radioactive isotope is incorporated such as 3H or 14C, are useful in distribution studies of substrates and/or drugs in tissues. Furthermore, the substitution of isotopes such as deuterium 2H may offer some therapeutic advantages derived from an improvement in metabolic stability. The isotopic variations of the agents of the invention may generally be prepared using conventional methods or with preparations described in the examples below using suitable isotopic variations of suitable reagents.

In the present context, the term “combination” refers to the accompaniment of EGCG with at least one of the following agents: folic acid, vitamin B12, low or very low molecular weight hyaluronic acid (1-500 kDa) to be co-administered in a subject as part of a given treatment regime. A treatment regime may include multiple and repeated joint-administrations of EGCG, folic acid, vitamin B12, low or very low molecular weight hyaluronic acid (1-500 kDa) for a predetermined period, for example for 1 month, for 2 months, for 3 months, for 4 months, for 5 months, for 6 months, for 7 months, for 8 months, for 9 months or more, a period of time in the range of 6 to 9 months is preferred. The co-administration of EGCG, folic acid, vitamin B12, low or very low molecular weight hyaluronic acid (1-500 kDa) may be repeated multiple times a day, for example once a day, twice a day, three times a day, four times a day, five times a day or more, a repetition of approximately once/twice a day is preferred. In other words, the co-administration of EGCG, folic acid, vitamin B12, low or very low molecular weight hyaluronic acid (1-500 kDa) is to be repeated for a predetermined amount of time, and a predetermined number of times a day which constitutes the prophylactic or therapeutic regimen.

The accompaniment of EGCG, folic acid, vitamin B12, low or very low molecular weight hyaluronic acid (1-500 kDa) understood as a “combination” of these substances need not be physical nor imply simultaneous administration. The reference to a “combination” of EGCG, folic acid, vitamin B12, low or very low molecular weight hyaluronic acid (1-500 kDa), therefore includes and describes multiple possible routes and times of administration of the respective substances.

Included and described in the term “combination” is for example the accompaniment of EGCG with at least one of the following agents: folic acid, vitamin B12, low or very low molecular weight hyaluronic acid (1-500 kDa) for the simultaneous administration by the same route, simultaneous administration by a different route, chronologically staggered administration (non-simultaneous) by the same route or chronologically staggered administration (non-simultaneous) by a different route. Each of routes of administration described here may be combined in any way, also if general it is preferable that the that the route be oral and/or topical application. Particularly preferable is the simultaneous administration of EGCG, folic acid, vitamin B12, low or very low molecular weight hyaluronic acid (1-500 kDa) for oral administration or for topical administration for example EGCG, folic acid, vitamin B12, low or very low molecular weight hyaluronic acid (1-500 kDa) are present in a composition, for example in one of the compositions exhibited here, for example under the form of a tablet, a hard capsule, a soft gel capsule, a powder, a syrup, a liquid to drink, a lozenge, an ointment, a cream, a gel.

The simultaneous administration of EGCG, folic acid, vitamin B12, low or very low molecular weight hyaluronic acid (1-500 kDa) for the same route of administration may for example include EGCG, folic acid, vitamin B12, low or very low molecular weight hyaluronic acid (1-500 kDa) in the same physical administered composition, for example by ingestion or applied topically by the subject, so that EGCG, folic acid, vitamin B12, low or very low molecular weight hyaluronic acid (1-500 kDa) enter into the body at the time by the same route of administration for example oral or topical. It is further possible, that also the combination of EGCG, folic acid, vitamin B12, low or very low molecular weight hyaluronic acid (1-500 kDa) for staggered administration (non-simultaneous) within a given co-administration is included and described in the term “combination”. The order of administration of EGCG, folic acid, vitamin B12, low or very low molecular weight hyaluronic acid (1-500 kDa) is not of particular importance, the timing of the staggered administration (non-simultaneous) of EGCG, folic acid, vitamin B12, low or very low molecular weight hyaluronic acid (1-500 kDa) may include prior administration of EGCG and the successive administration of at least one of the following agents: folic acid, vitamin B12, low or very low molecular weight hyaluronic acid or the prior administration of one of the following agents: folic acid, vitamin B12, low or very low molecular weight hyaluronic acid (1-500 kDa) followed by the successive administration of EGCG. For example, the staggered administration (non-simultaneous) of folic acid, vitamin B12, low or very low molecular weight hyaluronic acid (1-500 kDa) and EGCG for the same route of administration may oral and/or topical administration of at least one of the following agents folic acid, vitamin B12, low or very low molecular weight hyaluronic acid; accompanied with EGCG and at least one of the following agents: folic acid, vitamin B12, low or very low molecular weight hyaluronic acid (1-500 kDa) in this way it falls within the meaning of “combination” as described herein.

Staggered administration (non-simultaneous) of EGCG and at least one of the following agents: folic acid, vitamin B12, low or very low molecular weight hyaluronic acid (1-500 kDa) for different routes of administration, instead, may be for example comprised of initial topical administration of EGCG followed by administration of at least one of the following agents: folic acid, vitamin B12, low or very low molecular weight hyaluronic acid (1-500 kDa), in for example the form of a tablet; also the accompaniment of EGCG and at least one of the following agents: folic acid, vitamin B12, low or very low molecular weight hyaluronic acid (1-500 kDa) in this way also falls within the definition of “combination” as described herein.

In the present context “non-simultaneous” means EGCG and at least one of the following agents: folic acid, vitamin B12, low or very low molecular weight hyaluronic acid (1-500 kDa) are administered in a way that is chronologically staggered, at different times. The administration refers to a respective joint administration of EGCG and at least of the following agents: folic acid, vitamin B12, low or very low molecular weight hyaluronic acid (1-500 kDa) as part of a treatment of prophylactic regimen. For example, an embodiment predicts the administration of EGCG to the subject before the administration of at least one of the following agents: folic acid, vitamin B12, low or very low molecular weight hyaluronic acid (1-500 kDa) to the subject. Another embodiment predicts the administration of EGCG after the administration of at least one of the following agents: folic acid, vitamin B12, low or very low molecular weight hyaluronic acid (1-500 kDa) to the subject. It is understood that such non-simultaneous covers each respective instance of administration of EGCG and at least one of the following agents: folic acid, vitamin B12, low or very low molecular weight hyaluronic acid (1-500 kDa) in the context of a complete treatment regimen. As it mentioned within, independently of the order of administration i.e., EGCG first and at least one of the following agents: folic acid, vitamin B12, low or very low molecular weight hyaluronic acid (1-500 kDa), second or vice versa, the respective joint administration can be through the same or different routes of administration.

Another embodiment refers to EGCG and at least one of the following agents: folic acid, vitamin B12, low or very low molecular weight hyaluronic acid (1-500 kDa) are administered to a subject simultaneously. As mentioned above, the simultaneous administration of EGCG and at least one of the following agents: folic acid, vitamin B12, low or very low molecular weight hyaluronic acid (1-500 kDa) can be at administered through the same or different routes of administration. Typically, it is more advantageous and convenient to simultaneously administer EGCG and at least one of the following agents: folic acid, vitamin B12, low or very low molecular weight hyaluronic acid (1-500 kDa) through the same route of administration, preferably, through one route of oral/topical administration. It will be most commonly accomplished through combining EGCG and at least one of the following agents: folic acid, vitamin B12, low or very low molecular weight hyaluronic acid (1-500 kDa) administered in the same composition for example in the form of a tablet, including but not limited to an effervescent tablet, a powder (in particular presented in the form of a sachet), a hard capsule, a soft gel capsule, a syrup, a liquid for drinking, a lozenge, a pastille, or in the form of a ointment, a cream, a gel.

There are no particular restrictions on the duration of time between the administration of EGCG and at least one of the following agents: folic acid, vitamin B12, low or very low molecular weight hyaluronic acid (1-500 kDa) in the case of joint staggered administration i.e. non-simultaneous, but the it is generally more efficient and convenient if each time interval between the administration of EGCG and at least one of the following agents: folic acid, vitamin B12, low or very low molecular weight hyaluronic acid (1-500 kDa) is short, close to simultaneous, for example in the order of minutes. In some cases, such time intervals can extend to an hour or several hours between the respective administrations. In some cases, therefore, the joint administration of EGCG and at least one of the following agents: folic acid, vitamin B12, low or very low molecular weight hyaluronic acid (1-500 kDa) is chronologically staggered, the interval of time can be as long as 12 hours. Furthermore, also in the case in which EGCG and at least one of the following agents: folic acid, vitamin B12, low or very low molecular weight hyaluronic acid (1-500 kDa) are administered chronologically staggered, through the same or different routes of administration, the term “combination” means that a given joint administration of both EGCG and one of the following agents will have been completed: folic acid, vitamin B12, low or very low molecular weight hyaluronic acid (1-500 kDa) as part of a regimen, i.e. the substances will have been administered, before the joint administration of EGCG and at least one of the following agents: folic acid, vitamin B12, low or very low molecular weight hyaluronic acid (1-500 kDa) (which in turn may be administered simultaneously or chronologically staggered and through the routes of previous joint administrations) as part of the same regimen.

In the present context, the term “composition” includes and describes any physical entity including or constituting of or essentially constitution of the respective substances listed, for example including or consisting or essentially consisting of EGCG and at least one of the following agents: folic acid, vitamin B12, low or very low molecular weight hyaluronic acid (1-500 kDa). The physical form of the composition is not limited. For example, the term “composition” includes and describes a powder in which each of the listed substances is present in the form of a powder. As another example, the term “composition” includes and describes a liquid solution in which the listed substances are present in a solubilized form. As another example, the term “composition” also includes and describes an emulsion in which the listed substances are present. In another example, the term “composition” also includes and describes a suspension in which the listed substances are present. In another examples the term “composition” also includes and describes mixtures in which EGCG in a form, for example a powered solid, while one of at least of the following agents: folic acid, vitamin B12, low or very low molecular weight hyaluronic acid (1-500 kDa) is in another form for example a liquid. Specifically, the term “composition” may be a “pharmaceutical composition” as defined below and may be formulated for a route of administration. As it is used and described here, the term “composition” may therefore be a composition suitable for oral administration, for example in the form of a tablet, including, but not limited to an effervescent tablet or a multilayer tablet, a powder, for example in the form of a sachet, a hard capsule, a soft gel capsule, a syrup, a liquid to drink, a lozenge, a gummy lozenge, a balm, or other liquid preparation, an ointment, a cream, a gel. In some particularly preferred embodiments of the invention, the “composition” comprises EGCG and at least one of the following agents: folic acid, vitamin B12, low or very low molecular weight hyaluronic acid (1-500 kDa) and may be in the form of a soft gel capsule. In certain other embodiments, particularly preferred by the invention, the “composition” includes EGCG and at least one of the following agents: folic acid, vitamin B12, low or very low molecular weight hyaluronic acid (1-500 kDa) and may be in the form of a powder. In certain other preferred embodiments, the “composition” includes EGCG and at least one of the following agents: folic acid, vitamin B12, low or very low molecular weight hyaluronic acid (1-500 kDa) may be in the form of a cream, a gel, an ointment. The term “composition” may also be a composition suitable for a non-oral administration for example in the form of a suppository, a tablet, a hard capsule, a soft gel capsule, a cream, a gel, a plaster, a liquid. Other dosage forms of the composition in addition to their routes of administration are given below. The composition of the invention, including the pharmaceutical compositions, may therefore contain at least one pharmaceutically acceptable ingredient. While the inventive composition, including the pharmaceutical composition, can itself be administered to a subject, will be understood to add one or more pharmaceutically acceptable ingredients other than EGCG and at least one of the following agents: folic acid, vitamin B12, low or very low molecular weight hyaluronic acid (1-500 kDa) can be advantageous in making the composition more suitable for direct administration by a given predetermined route. Consequently, the “pharmaceutical composition” may be formulated to contain EGCG and one of the following agents: folic acid, vitamin B12, low or very low molecular weight hyaluronic acid (1-500 kDa), a pharmaceutically acceptable ingredient that makes the composition more suitable or particularly suitable for the direct administration by a given predetermined route, without further testing. This suitability may regard a series of different routes of administration, such as oral, parenteral, transmucosal, vaginal or perivaginal, topical, transdermal or intravesical, as further explained below. Where present, and by way of non-limiting illustration, such ingredients, and their positive impact on the suitability of the inventive composition for diverse routes of administration are reported further below.

### a) Formulation Suitable for Oral Administration

A composition suitable for oral administration may be prepared, packaged or sold in the form of an individual solid dose which includes a sachet (for example a powder in a sachet), a tablet, a hard or soft capsule, each containing a predetermined quantity of EGCG and at least one of the following agents: folic acid, vitamin B12, low or very low molecular weight hyaluronic acid (1-500 kDa) as it is here specified. Other formulations suitable for oral administration include a powder or granular form, an aqueous or oily suspension, an aqueous or oily solution, or an emulsion. In the present context, an “oily” liquid comprises of a carbon containing liquid molecule which is less polar in respect to water.

It is particularly preferred that an individual solid dose of the inventive composition is in the form of a powder, presented in the form of a sachet. In the present context, the term “sachet” refers to a sealed bag containing the inventive composition. The bag may be made of paper, waxed paper, laminated paper, or a combination of a paper and foil. The material of which the sachet is made from is preferably, impermeable to ambient humidity and other potential environmental contaminants so that by sealing the sachet the inventive composition remains as a powder in a free-flowing form until its use.

A tablet containing EGCG and at least of the following agents: folic acid, vitamin B12, low or very low molecular weight hyaluronic acid (1-500 kDa) in any of the concentration listed above can, for example be made by compressing EGCG and at least one of the following agents: folic acid, vitamin B12, low or very low molecular weight hyaluronic acid (1-500 kDa) with one or more added ingredients. The tablets, obtained by compression, can be obtained by compressing, in a suitable device, EGCG and at least of the following agents: folic acid, vitamin B12, low or very low molecular weight hyaluronic acid (1-500 kDa) in a free-flowing powder or granular form optionally mixed with one or more of a binder, a lubricant, an excipient, a surfactant, and a dispersing agent. Moulded tablets can be obtained by moulding, can be obtained by moulding, in a suitable device, EGCG and at least of the following agents: folic acid, vitamin B12, low or very low molecular weight hyaluronic acid (1-500 kDa), a pharmaceutically acceptable vehicle and liquid to sufficiently moisten the mixture.

Pharmaceutically acceptable excipients used in the production of tablets include but are not limited to inert diluents, granulating and disintegrating agents, binding agents, and lubricating agents. The dispersing agents of note include but are not limited to, potato starch and sodium starch glycollate. The surfactants of note include, but are not limited to, sodium lauryl sulfate. The thinners of note include, but are not limited to calcium carbonate, sodium carbonate, lactose, microcrystalline cellulose, calcium phosphate, calcium hydrogen phosphate, and sodium phosphate. The granulating and disintegrating agents of note include, but are not limited to, corn starch and alginic acid. The binding agents of note include, but are not limited to, gelatine, acacia, pre-gelatinized corn starch, polyvinylpyrrolidone and hydroxypropyl methylcellulose. Lubricating agents of note include, but are not limited to, magnesium stearate, stearic acid, silica, and talcum powder.

The tablets may be uncoated or coated with methods known to achieve delayed disintegration in the gastrointestinal tract of a subject, thereby providing a prolonged release and absorption of EGCG and at least one of the following agents: folic acid, vitamin B12, low or very low molecular weight hyaluronic acid (1-500 kDa). For example, to coat the tablets a material such as glyceryl monostearate or glyceryl distearate may be used. The tablets may therefore contain a sweeting agent, an aromatizing agent, a colouring agent, a preservative, or some combination to provide a pharmaceutically interesting and acceptable preparation.

The hard capsules that contain EGCG and at least one of the following agents: folic acid, vitamin B12, low or very low molecular weight hyaluronic acid (1-500 kDa) may be obtained using a physiological degradable composition, such as gelatine or cellulose derivates. These hard capsules that contain EGCG and at least one of the following agents: folic acid, vitamin B12, low or very low molecular weight hyaluronic acid (1-500 kDa) may therefore contain additional ingredients that include, for example an inert solid diluent such as calcium carbonate, calcium phosphate or kaolin.

Soft gelatine capsules containing EGCG and at least one of the following agents: folic acid, vitamin B12, low or very low molecular weight hyaluronic acid (1-500 kDa) may be obtained using a physiological degradable composition, such as gelatine combined with a plasticizer (i.e., glycerol) as a base component of a soft gelatine capsule. The soft gelatine capsule may contain a preconcentrated solution or suspension or liquid microemulsion or semisolid. The inside of the soft capsule containing EGCG and at least one of the following agents: folic acid, vitamin B12, low or very low molecular weight hyaluronic acid (1-500 kDa), may be mixed with water or an oily medium such as peanut oil, liquid paraffin, olive oil, soybean oil, sunflower oil, a lecithin such as soy lecithin or sunflower lecithin, medium chain triglycerides, polyglycerol oleate, beeswax, mono- and diglycerides of fatty acids or any of the forementioned in any combination.

Liquid formations that are partially suitable for oral administration may be prepared, packaged and sold in liquid form or in a solid form designed to be reconstituted with water or other vehicles suitable for ingestion. The liquid suspensions may be prepared with conventional methods for obtaining the suspension of one or more active ingredients in an aqueous or oily vehicle. Aqueous vehicles include, for example, water and isotonic saline. Oily vehicles contain, for example, almond oil, oily esters, ethyl alcohol, vegetable oils such as peanut, olive, sesame or coconut oil, fractionated vegetable oils and mineral oils such as liquid paraffin.

The liquid suspensions may therefore contain one or more additional ingredients including, but not limited to suspending agents, dispersing, or wetting agents, emulsifying agents, emollients, preservatives, buffers, salts, flavours, colouring agents, and sweetening agents. The oily suspensions further contain a thickening agent. The suspending agents of note include but are not limited to sorbitol syrup, hydrogenated edible fats, sodium alginate, polyvinylpyrrolidone, tragacanth gum, acacia gum and cellulose derivatives such as sodium carboxymethylcellulose, methylcellulose, hydroxypropyl methylcellulose. The dispersing or wetting agents of note include but are not limited to naturally occurring phosphatides such as lecithin, condensation products of an alkylene oxide with a fatty acid, with a long-chain aliphatic alcohol, with a partial ester derived from a fatty acid and a hexyl, or with a partial ester derived from a fatty acid and a hexyl anhydride (e.g., polyoxyethylene stearate, heptadecaethyleneoxycethanol, polyoxyethylene sorbitol monooleate, and polyoxyethylene sorbitan monooleate, respectively). Emulsifying agents of note include but are not limited to lecithin and acacia. Preservatives of note include but are not limited to methyl, ethyl, or n-propyl para-hydroxybenzoates, ascorbic acid and sorbic acid. Sweeting agents of note include, for example glycerol, propylene glycol, sorbitol, sucrose, and saccharin. The thickening agents of note for oily suspensions include, for example, beeswax, paraffin wax and cetyl alcohol. The powder or granular powder formulations of a composition suitable for the application in the present invention may be prepared with known methods. Such formulations may be directly administered to a subject, using, for example, a sachet or tablets, for filling capsules or for preparing a suspension, or aqueous or oily solution added to an aqueous or oily vehicle. Each of these formations may include or more dispersing or wetting agents, a suspending agent, and a preservative. In these formulations additional excipients, such as fillers and sweetening, aromatizing, and colouring agents may be included. A composition may also be prepared, packaged, and sold in the form of an emulsion of oil in water or an emulsion of water in oil. The oily phase may be a vegetable oil such as olive or peanut oil, or a mineral oil such as liquid paraffin or a combination of these. Such compositions may further contain one or more emulsifying agents such as naturally present rubbers such as gum acacia or gum tragacanth, naturally occurring phosphatides such as soy phosphatide or lecithin, esters or partial esters derived from combinations of fatty acids and hexyl anhydrides such as sorbitan monooleate, and condensation products of such partial esters with ethylene oxide such as polyoxyethylene sorbitan monooleate. These emulsifiers may contain addition ingredients such as for example, sweetening or aromatizing agents. Methods of inserting or coating a material with a chemical composition are known in the art, include but are not limited to, methods of depositing or bonding a chemical composition onto a surface, methods of incorporating a chemical composition in the structure of a material during the synthesis of the material (for example with a physiologically degradable material) and methods of absorbing a solution, or aqueous or oily suspension into an absorbent material with or without subsequent drying.

### b) Formulation Suitable for Parenteral Administration

For parenteral administration, a composition containing EGCG and at least one of the following agents: folic acid, vitamin B12, low or very low molecular weight hyaluronic acid (1-500 kDa) may be formulated for injection or infusion for example injection or intravenous infusion, intramuscular or subcutaneous or for administration as a bolus dose and/or continuous infusion. Suspensions, solutions, and emulsions may be used in an oily or aqueous vehicle, optionally containing other agents such as suspending, stabilizing and/or dispersing agents as mentioned above.

A composition containing EGCG and at least one or more of the following agents: folic acid, vitamin B12, low or very low molecular weight hyaluronic acid (1-500 kDa) may be made particularly suitable for parenteral administration by using a pharmaceutically acceptable carrier, such as sterile water or isotonic saline. These formulations may be prepared, packaged, and sold in a form suitable for administration as a bolus dose or continuous administration. The injectable formulations may be prepared, packaged, as a single-unit dose for example in ampoules, unbreakable or not, or in multi-dose containing a preservative. Compositions particularly suitable for parenteral administration included but are not limited to suspensions, solutions, emulsions in an oily or aqueous vehicle, extended release or biodegradable pastes and implantable formulations. These compositions may further include one or more added ingredients including but not limited to, suspending, stabilizing, or dispersing agents. In a further embodiment of a composition particularly suitable for parenteral administration, the active ingredient is provided in dry form (for example as a power or granulated powder) for the reconstitution with a suitable vehicle (for example sterile pyrogen free water) before parenteral administration of the reconstituted composition. A composition suitable for the application in the present invention may be prepared, packaged, and sold in the form of a sterile injectable aqueous or oily suspension or solution. This suspension or solution may be formulated according to the known art and can contain, in addition one or more active ingredients, added ingredients such as dispersing, wetting or suspending agents described herein. Such sterile injectable compositions for parenteral routes of administration include but are not limited to Ringer's solution, isotonic sodium chloride solution, and fixed oils such as synthetic mono- or diglycerides. Other common formulations for parenteral routes of administration include those that contain the active ingredient in a microcrystalline form, in a liposomal preparation or as part of a biodegradable polymeric system.

Compositions for sustained release or implantation can include polymeric or pharmaceutically acceptable hydrophobic materials such as an emulsion, an ion exchange resin, a sparingly soluble polymer, or a sparingly soluble salt.

### c) Formulation Suitable for Transmucosal Administration

A composition including EGCG and at least one of the following agents: folic acid, vitamin B12, low or very low molecular weight hyaluronic acid (1-500 kDa) may be formulated to be suitable for transmucosal administration. The formulation may include any substances or dosage unit suitable for application to mucosal tissue. For example, selected active agents, may be administered in the buccal mucosa in an adhesive tablet or a patch, administered sublingually as a solid dose under the tongue, administered lingually by placing a solid dose on the tongue, administered nasally as drops or a nasal spray, a liquid non aerosol formulation or a dry powder, placed insider or near the rectum (transrectal formulations) or administered through the urethra as a suppository, ointment or similar.

### d) Formulation Suitable for Vaginal or Perivaginal Administration

A composition including EGCG and at least one of the following agents: folic acid, vitamin B12, low or very low molecular weight hyaluronic acid (1-500 kDa) may be formulated to be suitable for vaginal or perivaginal administration. Suitable dosage units for this application include vaginal suppositories, creams, ointments, liquid formulations, vaginal pessaries, tampons, gels, pastes, foams, or sprays. The suppository, cream, ointment, liquid formulation, vaginal pessary, tampon, gel, paste, foam, or spray for vaginal or perivaginal release includes a quantity of therapeutic efficacious active agent or selected active agents and one or more non-toxic conventional carriers for vaginal or perivaginal drug administration. The vaginal or perivaginal administration in the present invention may be produced with conventional methods as described, for example, in Remington: The Science and Practice of Pharmacy. The vaginal or perivaginal dosage unit may be a product for rapid disintegration or for a period of several hours. The period of time for the complete disintegration may be an interval from approximately 10 minutes to 6 hours, for example less than approximately 3 hours.

### e) Formulation Suitable for Topical Administration

A composition including EGCG and at least one of the following agents: folic acid, vitamin B12, low or very low molecular weight hyaluronic acid (1-500 kDa) may also be formulated to be suitable for topical administration. Dosage forms adapted for this application include forms suitable for application to the surface of the body and may include, for example, an ointment, cream, gel, lotion, solution, paste and the like, and/or they may be prepared to contain liposomes, micelles and/or microspheres. In some embodiments the topical formulations here reported are ointments, creams or gels.

### f) Formulation Suitable for Transdermal Administration

A composition including EGCG and at least one of the following agents: folic acid, vitamin B12, low or very low molecular weight hyaluronic acid (1-500 kDa) may be formulated to be suitable for transdermal administration. As it is known to someone skilled in the art, transdermal administration involves the release of pharmaceutical compounds through the percutaneous passage, allowing transport of the compound into systemic circulation of the patient. This may be achieved, for example, by transdermal patches or using iontophoresis devices. The transdermal patches may, for example, also incorporate other components other than EGCG and at least one of the following agents: folic acid, vitamin B12, low or very low molecular weight hyaluronic acid (1-500 kDa). For examples compositions and/or transdermal patches may include formulations with one or more preservatives or bacteriostatic agents including, but not limited to, methyl hydroxybenzoate, propyl hydroxybenzoate, chlorocresol, benzalkonium chloride and the like. Dosage forms of the inventive composition for the topical administration of EGCG at least one of the following agents: folic acid, vitamin B12, low or very low molecular weight hyaluronic acid (1-500 kDa) may include creams, sprays, lotions, gels, ointments, eye drops, nasal drops, ear drops and the like. In such dosage forms, ingredients of the inventive composition may be mixed to form a smooth homogeneous, white opaque cream or lotion, examples include 1% or 2% (w/w) benzyl alcohol as a preservative, emulsifying wax, glycerine, isopropyl palmitate, lactic acid, purified water, and sorbitol solution. Further, the composition may contain polyethylene glycol 400. They may be mixed to form a lotion with, for example, 2% (w/w) benzyl alcohol as a preservative, white petrolatum, emulsifying wax and Tenox II (butylated hydroxyanisole, propyl gallate, citric acid, propylene glycol).

Tampons or rolls of dressing material, for example gauze, in which the composition may be inserted in the form of a solution, lotion, cream, ointment or the like, may be used also for topical application. The compositions may be applied both topically by means of a transdermal system, as an acrylic-based polymeric adhesive with a resinous crosslinking agent containing the composition and laminated onto an impermeable backing. Examples of suitable skin-contact adhesive materials include, but not are limited to, polyethylenes, polysiloxanes, polyisobutylenes, polyacrylates, polyurethanes and the like. As an alternative, the reservoir which contains the drug and the skin-contact adhesive are separate and distinct, with the adhesive underlining the reservoir, which, in this case, may be a polymer matrix as described above or be a liquid or hydrogel reservoir, or it may take some other form.

### g) Formulation Suitable for Intravesical Administration

The term intravesical administration as it is used here in conventional sense indicates the release of a drug directly into the bladder. Methods suitable for intravesical administration may be found, for example, in U.S. Pat. No. 6,207,180 e 6,039,967.

The combination of EGCG and at least one of the following agents: folic acid, vitamin B12, low or very low molecular weight hyaluronic acid (1-500 kDa) may be administered according to the uses and methods describes here as a dietary product or a drink containing EGCG and at least one of the following agents: folic acid, vitamin B12, low or very low molecular weight hyaluronic acid (1-500 kDa). In the present context, the term “dietary product” refers to an ingestible substance that, at the temperature at which it is correctly stored and ingested, is a solid or semi solid, and that will be chewed prior to swallowing. In the present context, the term “drinkable product” refers to an ingestible that, at the temperature in which it is correctly stored and injected, is a free-flowing liquid that will not be chewed prior to swallowing. In principle, the dietary product, may be any dietary product that has been treated to contain the combination of EGCG and at least one of the following products: folic acid, vitamin B12, low or very low molecular weight hyaluronic acid (1-500 kDa) to be administered. In the case in which the combination of EGCG and at least one of the following agents: folic acid, vitamin B12, low or very low molecular weight hyaluronic acid (1-500 kDa) must be administered, the dietary product will contain EGCG and least one of the following agents: folic acid, vitamin B12, low or very low molecular weight hyaluronic acid (1-500 kDa) in the expected concentration. Examples of such dietary products include dietary bars, such as a chocolate bar, a muesli bar, an ice cream bar or an energy bar; a chewing gum; a sweet; a mint; a yogurt; an edible gel; a ready meal, for example a free-dried ready meal; spreadable cream; a pudding; or a processed fruit product such as a fruit roll or fruit stick. Examples of such drinkable products include fruit juice, or drinks containing fruit juice, milk-based drinks, for example drinks containing milk or drinks containing buttermilk, drinks containing whey, yogurt drinks, energy drinks, non-alcoholic drinks, flavoured water drinks etc. The applicant has found that the inclusion of a certain minimum quantity of EGCG and at least one of the following agents: folic acid, vitamin B12, low or very low molecular weight hyaluronic acid (1-500 kDa) in the inventive composition may be advantageous in guaranteeing the desired therapeutic or prophylactic effect and may be obtained with a reasonable number of administrations. Typically, it is possible to achieve the prevention, the control and/or inhibition of HPV, with a daily administration of combined total of approximately 200 mg of EGCG, approximately 400 μg of folic acid, approximately 1 mg of vitamin B12, approximately 50 mg of low or very low molecular weight hyaluronic acid (1-500 kDa).

However, from the point of view of the impact of such repeated administrations on the quality of life of the patient, it may be advantageous to guarantee a minimum quantity of EGCG and at least one of the following products: folic acid, vitamin B12, low or very low molecular weight hyaluronic acid (1-500 kDa) corresponding to the number of daily administrations necessary for achieving the target of quantity of EGCG and at least one of the following agents: folic acid, vitamin B12, low or very low molecular weight hyaluronic acid (1-500 kDa), for example the target quantity of EGCG and at least one of the following agents: folic acid, vitamin B12, low or very low molecular weight hyaluronic acid (1-500 kDa) indicated above.

Therefore, an embodiment of the uses and methods described here may provide the administration of approximately 200 mg of EGCG, approximately 400 μg of folic acid, approximately 1 mg of vitamin B12, approximately 50 mg of low or very low molecular weight hyaluronic acid (1-500 kDa) in total as a combined administration in the subject. Another embodiment of the uses and methods described here may provide the administration of approximately 100 mg of EGCG, approximately 200 μg of folic acid, approximately 0.5 mg of vitamin B12, approximately 25 mg of low or very low molecular weight hyaluronic acid (1-500 kDa) in total as a combined administration in the subject. Another embodiment of the uses and methods described here may provide the administration of approximately 65 mg of EGCG, approximately 130 μg of folic acid, approximately 330 μg of vitamin B12, approximately 15 mg of low or very low molecular weight hyaluronic acid (1-500 kDa) in total as a combined administration in the subject. Another embodiment of the uses and methods described here may provide the administration of approximately 50 mg of EGCG, approximately 100 μg of folic acid, approximately 250 μg of vitamin B12, approximately 12.5 mg of low or very low molecular weight hyaluronic acid (1-500 kDa) in total as a combined administration in the subject (for example in the form of a powder, for example in the form of a sachet.) Another embodiment of the uses and methods described here may provide the administration of approximately 40 mg of EGCG, approximately 80 μg of folic acid, approximately 200 μg of vitamin B12, approximately 10 mg of low or very low molecular weight hyaluronic acid (1-500 kDa) in total as a combined administration in the subject. For example, such medical uses and methods of treatment may provide the administration of approximately 30 mg of EGCG, approximately 60 μg of folic acid, approximately 140 μg of vitamin B12, approximately 7 mg of low or very low molecular weight hyaluronic acid (1-500 kDa) in total as a combined administration in the subject.

Another embodiment of the uses and methods described here may provide the administration of approximately 800 mg of EGCG, approximately 400 μg of folic acid, approximately 1000 μg of vitamin B12, approximately 200 mg of low or very low molecular weight hyaluronic acid (1-500 kDa) in total as a combined administration in the subject.

Another embodiment of the uses and methods described here may provide the administration of approximately 600 mg of EGCG, approximately 400 μg of folic acid, approximately 1000 μg of vitamin B12, approximately 200 mg of low or very low molecular weight hyaluronic acid (1-500 kDa) in total as a combined administration in the subject.

An embodiment of the uses and methods described here may provide the administration of approximately 200 mg of EGCG, approximately 400 μg of folic acid, approximately 1000 μg of vitamin B12, approximately 50 mg of low or very low molecular weight hyaluronic acid (1-500 kDa) total or between 50-300 mg of EGCG, 200-400 μg of folic acid, 500-1000 μg of vitamin B12, and 100-200 mg of low or very low molecular weight hyaluronic acid (1-500 kDa) in any administration, for example in any combined administration in a subject. An embodiment of the uses and methods described here may provide the administration of between 1-800 mg of EGCG, 1-400 g of folic acid, 1-1000 μg of vitamin B12, and 1-200 mg of low or very low molecular weight hyaluronic acid (1-500 kDa) in any administration, for example in any combined administration in the subject.

Another embodiment of the uses and methods described here may provide the administration of approximately 200 mg of EGCG, approximately 400 μg of folic acid, approximately 1000 μg of vitamin B12, approximately 50 mg of low or very low molecular weight hyaluronic acid (1-500 kDa) in any administration, for example in any combined administration in a subject.

Another embodiment of the uses and methods described here may provide the administration of approximately 800 mg of EGCG, approximately 400 μg of folic acid, approximately 1000 μg of vitamin B12, approximately 200 mg of low or very low molecular weight hyaluronic acid (1-500 kDa) in any administration, for example in any combined administration in a subject.

An embodiment in the case in which the combination of EGCG, folic acid, vitamin B12, low or very low molecular weight hyaluronic acid (1-500 kDa) must be administered to the subject in the form of a powder, for example in a sachet, it may include or be constituted of approximately 200 mg of total EGCG, approximately 400 μg of total folic acid, around 1000 μg of total vitamin B12 and around 50 mg of total low or very low molecular weight hyaluronic acid (1-500 kDa), and this may be administered to the subject. A related embodiment in the total combination of EGCG, folic acid, vitamin B12, low or very low molecular weight hyaluronic acid (1-500 kDa) may be administered to the subject in the form of a powder (for example as a sachet), and may include approximately 100 mg of total EGCG, approximately 200 μg of total folic acid, around 500 μg of total vitamin B12 and around 25 mg of total low or very low molecular weight hyaluronic acid (1-500 kDa) and this may be administered to the subject. An alternative embodiment in the case in which the combination of EGCG, folic acid, vitamin B12, low or very low molecular weight hyaluronic acid (1-500 kDa) must be administered to the subject in the form of a soft gel capsule, it may include or be constituted of approximately 200 mg of total EGCG, approximately 400 μg of total folic acid, around 1000 μg of total vitamin B12 and around 50 mg of total low or very low molecular weight hyaluronic acid (1-500 kDa), and this may be administered to the subject. A related embodiment in the total combination of EGCG, folic acid, vitamin B12, low or very low molecular weight hyaluronic acid (1-500 kDa) may be administered to the subject in the form of a liquid gel capsule, and may include approximately 100 mg of total EGCG, approximately 200 μg of total folic acid, around 500 μg of total vitamin B12 and around 25 mg of total low or very low molecular weight hyaluronic acid (1-500 kDa) and this may be administered to the subject.

Above all these final embodiments contain the administration of approximately 200 mg of total EGCG, approximately 400 μg of total folic acid, around 1000 μg of total vitamin B12 and around 50 mg of total low or very low molecular weight hyaluronic acid (1-500 kDa), or the administration of approximately 100 mg of total EGCG, approximately 200 μg of total folic acid, around 500 μg of total vitamin B12 and around 25 mg of total low or very low molecular weight hyaluronic acid (1-500 kDa), they are particularly advantageous because such combined administrations are necessary once or twice a day, respectively, in order to reach the daily target quantity. The following quantities and combinations represent the embodiments preferred by the present invention:
Approximately 1-800 mg EGCG and approximately 1-400 μg of folic acid
Approximately 50-600 mg EGCG and approximately 50-400 μg of folic acid
Approximately 100-300 mg EGCG and approximately 200-400 μg of folic acid
Approximately 160-240 mg EGCG specifically approximately 200 mg EGCG+20%
Approximately 400 μg of folic acid
Approximately 1-800 mg EGCG and approximately 1-1000 μg of vitamin B12
Approximately 50-600 mg EGCG and approximately 100-1000 μg of vitamin B12
Approximately 100-300 mg EGCG and approximately 500-1000 μg of vitamin B12
Approximately 1000 μg of vitamin B12.
Approximately 1-800 mg EGCG and approximately 1-200 mg of low or very low molecular weight hyaluronic acid (1-500 kDa)
Approximately 50-600 mg EGCG and approximately 25-150 mg of low or very low molecular weight hyaluronic acid (1-500 kDa).
Approximately 100-300 mg EGCG and approximately 50-100 mg of low or very low molecular weight hyaluronic acid (1-500 kDa)
Approximately 50 mg of low or very low molecular weight hyaluronic acid (1-500 kDa)
Approximately 800 mg of EGCG, approximately 400 μg of folic acid, approximately 1000 μg of vitamin B12 and approximately 200 mg of low or very low molecular weight hyaluronic acid (1-500 kDa)
Approximately 600 mg of EGCG, approximately 400 μg of folic acid, approximately 1000 μg of vitamin B12 and approximately 200 mg of low or very low molecular weight hyaluronic acid (1-500 kDa)
Approximately 100 mg of EGCG, approximately 200 μg of folic acid, approximately 500 μg of vitamin B12 and approximately 25 mg of low or very low molecular weight hyaluronic acid (1-500 kDa)
Approximately 50 mg of EGCG, approximately 100 g of folic acid, approximately 250 μg of vitamin B12 and approximately 12.5 mg of low or very low molecular weight hyaluronic acid (1-500 kDa)
Approximately 200 mg of EGCG, approximately 400 μg of folic acid, approximately 1000 μg of vitamin B12 and approximately 50 mg of low or very low molecular weight hyaluronic acid (1-500 kDa)

The following provides various examples that illustrate the various embodiments of the present invention and the technical effects and advantages. It should be understood that the following presented examples are only illustrative and do not limit the claimed invention. In fact, a skilled person may easily recognize the other embodiments in the spirit and scope of the claimed invention, while also understanding said technical advantages.

### EXAMPLES

The present invention provides a combination and/or composition of EGCG with at least one of the following agents: folic acid, vitamin B12, low or very low molecular weight hyaluronic acid (1-500 kDa) in order to give a more advantageous effect in respect to the use of the single substances in the prevention, control, and inhibition of the HPV infection. This represents a new and unexpected approach in the prevention/treatment of the HPV infection.

### Example 1

A first exemplary composition in the present invention is the form of an oblong tablet (19×8 mm in diameter), approximately 900 mg in weight, with a reported storage period of 24 months (if stored in a cool, dry, dark place) and having the following composition:

TABLE 1
Composition of example 1

| | | Contribution |
|---|---|---|
| Ingredient | mg/tablet | per tablet |
| Green TEA leaves E.S. 45% EGCG | 444.5 | 200 mg |
| | | of EGCG |
| SODIUM HYALURONATE (3-10 KDa) | 57.50 | 50 mg |
| 86.95% HYALURONIC ACID | | |
| CYANOCOBALAMIN (VIT B12) 100% | 1 | 1 mg |
| 5-MTHF 88% | 0.45 | 0.4 mg |
| | | of folate |
| Microcrystalline cellulose | 329.55 | |
| Silicon dioxide | 20 | |
| Magnesium stearate | 20 | |
| Hydroxypropylcellulose | 27 | |
| Total | 900 mg | |

It can be noted in the present example, the tolerance interval for the titre of EGCG is 160-240 mg/tablet (200±20%).

The amount of ingredients reported in the column “mg/tablet” of Table 1 refers to the amount of ingredient used for preparing a tablet. The amount of ingredients reported in the column “contribution per tablet” of Table 1 refers to the amount of nutrient present in the tablet. The excessive dosage of 5-MTHF and sodium hyaluronate are related to the following factors:
Titre of raw starting material
Chemical form of the starting molecular substrate
Loss of substance during the process of production.

One tablet a day is recommended.

### Example 2

A second exemplary composition in the present invention is the form of an oblong tablet (19×8 mm in diameter), approximately 900 mg in weight, with a reported storage period of 24 months (if stored in a cool, dry, dark place) and having the following composition:

TABLE 2
Composition of example 2

| | | Contribution |
|---|---|---|
| Ingredient | mg/tablet | per tablet |
| Green TEA leaves E.S. 95% | 444.5 | 200 mg |
| polyphenols 45% EGCG | | of EGCG |
| SODIUM HYALURONATE (3-10 KDa) | 57.50 | 50 mg |
| 86.95% HYALURONIC ACID | | |
| CYANOCOBALAMIN (VIT B12) 100% | 1 | 1 mg |
| L-5-methylfolate 88% | 0.45 | 0.4 mg |
| | | of folate |
| Microcrystalline cellulose | 329.55 | |
| Silicon dioxide | 20 | |
| Magnesium stearate | 20 | |
| Hydroxypropylcellulose | 27 | |
| Total | 900 mg | |

It can be noted in the present example, the tolerance interval for the titre of EGCG is 160-240 mg/tablet (200±20%)

The amount of ingredients reported in the column “mg/tablet” of Table 2 refers to the amount of ingredient used for preparing a tablet. The amount of ingredients reported in the column “contribution per tablet” of Table 2 refers to the amount of nutrient present in the tablet. The excessive dosage of L-5-methylfolate and sodium hyaluronate are related to the following factors:
Titre of raw starting material
Chemical form of the starting molecular substrate
Loss of substance during the process of production.

One tablet a day is recommended.

### Example 3—In Vitro Experiments

### Experimental Design

The combined action of epigallocatechin gallate (EGCG) and at least one the following other agents: folic acid, vitamin B12, hyaluronic acid, in respect to the single substance, has been tested in vitro in immortalized cervical cancer cell line (HeLa).

The cells were seeded in 25 cm³ flasks in low glucose DMEM, to which had been added FBS (Fetal Bovine Serum) 10%, L-Glu (L-glutamine) 1%, Pen/Strep (penicillin/streptomycin) 1%. The cells were kept in an incubator at a temperature of 37° C., 5% carbon dioxide and 95% humidity and maintained in culture, as described above, until they reached 50% confluency. On the day of the experiment the cells were treated as follows:
1. Control (cells in culture medium without additional stimulus
2. EGCG 50 mcg/ml
3. Calcium L-methylfolate (900 nM)
4. Vitamin B12 (1000 nM)
5. Hyaluronic acid (10 mcg/ml)
6. Combined treatment (EGCG+folic acid; EGCG+vitamin B12; EGCG+hyaluronic acid)
7. Combined treatment (EGCG+folic acid+vitamin B12+hyaluronic acid

EGCG was used in the form of green tea extract with polyphenol titre of 95% and an EGCG content of 45%. The given concentration refers to the active ingredient EGCG.

Apoptosis (cell death) was evaluated after 48 hours of treatment using the Annexin V kit. The expression of onco-suppressor genes (p53 and/or pRB) and/or viral E6 and/or E7 genes was evaluated after 48 hours of treatment through use of real time PCR. A statistical analysis was performed.

### Cell Culture

The human Hela cervical carcinoma cell line was obtained from European Collection of Cell Cultures (ECACC). The cells were seeded into 25 cm² flasks (Falcon, Becton Dickinson Labware, Franklin Lakes, NJ, USA), grown in monolayer culture in a 1:1 mixture of Dulbecco's modified Eagle's Minimal Essential Medium (DMEM) and Ham's F12 medium containing 5% fetal bovine serum (FBS), antibiotics (penicillin 100 IU/mL, streptomycin 100 μg/mL, gentamycin 200 μg/mL all from Euroclone Ltd, Cramlington, UK). The cells were cultured at 37° C. in air with 5% CO₂. The medium was changed every three days. At confluence, the cells were subcultured after removal with 0.05% trypsin-0.01% EDTA.

### Annexin V/7-AAD Staining

Hela cells were cultured at confluence into 25 cm² flasks (Falcon, Becton Dickinson Labware, Franklin Lakes, NJ, USA) in complete medium. Then, the medium was replaced by fresh complete medium containing EGCG (50 mcg/ml) or folic acid (Fol) (900 nM) or vitamin (B12) (1000 nM) or low or very low molecular weight hyaluronic acid (Hyal) (10 mcg/ml) or containing EGCG (50 mcg/ml)+Fol (900 nM)+B12 (1000 nM)+Hyal (10 mcg/ml). After 48 hr of treatment, cell status was assessed through use of the FITC Annexin V Apoptosis Detection Kit I according to the instructions of the manufacturer (BD Pharmingen™).

Expression of p53, E6/E7 and Rb Messenger RNA

Hela cells were cultured at confluence in 25 cm² flasks (Falcon, Becton Dickinson Labware, Franklin Lakes, NJ, USA) in a complete medium. Then, after 24 hr, the medium was replaced by fresh complete medium containing EGCG (50 mcg/ml) or Fol (900 nM) or B12 (1000 nM) or Hyal (10 mcg/ml) or containing EGCG (50 mcg/ml)+Fol (900 nM)+B12 (1000 nM)+Hyal (10 mcg/ml). After 48 hr of treatment, the total RNA was isolated using the RNeasy Plus Mini Kit (Qiagen, Germany) according to manufacturer instructions. 1 μg of total RNA was reverse transcribed into complementary (cDNA) using Fast Gene Scriptase II cDNA kit (Nippon Genetics Europe, Germany). 1 μg of total cDNA was used for qPCR using the iTaq Universal SYBR Green Supermix (BioRad, Hercules, CA, USA). mRNA levels were standardized using β-actin cDNA. The ratio was compared between treated and control conditions and the analysis performed in triplicate. The results were expressed as fold change with respect to control values. Commercially available primers for qRT-PCR of Rb, E6/E7 mRNA, and β-actin were used as previously published.

•p53:
Forward
(SEQ ID No. 1)
5′-ACATGACGGAGGTTGTGA-3′
Reverse
(SEQ ID No. 2)
5′-CGCAAATTTCCTTCCACTC-3′;
•Rb:
Forward
(SEQ ID No. 3)
5′-GCTAGCCTATCTCCGGCTAAA-3′
and
Reverse
(SEQ ID No. 4)
5′-CTGGAAAAGGGTCCAGATGA-3′;
•E6/E7:
Forward
(SEQ ID No. 5)
5′-ATGCATGGACCTAAGGCAAC-3′
and
Reverse
(SEQ ID No. 6)
5′-AGGTCGTCTGCTGAGCTTTC-3′;
•β-actin:
Forward
(SEQ ID No. 7)
5′-CAACCGCGAGAAGATGACC-3′
and
Reverse
(SEQ ID No. 8)
5′-AGAGGCGTACAGGGATAGCA-3′;

(Darnell G A, Antalis T M, Johnstone R W, Stringer B W, Ogbourne S M, Harrich D, Suhrbier A. Inhibition of retinoblastoma protein degradation by interaction with the serpin plasminogen activator inhibitor 2 via a novel consensus motif. Mol Cell Biol. 2003 September; 23 (18): 6520-32. doi: 10.1128/MCB.23.18.6520-6532.2003) (Fuso A, Cavallaro R A, Zampelli A, D'Anselmi F, Piscopo P, Confaloni A, Scarpa S. Gamma-Secretase is differentially modulated by alterations of homocysteine cycle in neuroblastoma and glioblastoma cells. J Alzheimers Dis 2007; 11:275-90. doi: 10.3233/jad-2007-11303).

### Statistical Analysis

Statistical analysis GraphPad Prism 8 (GraphPad, La Jolla, CA, USA) was used for statistical and graphical analyses. One-way ANOVA was calculated, and the Bonferroni post-test was used to evaluate any significant (p<0.05) difference reported in this paper. The assays were repeated in triplicate and the histograms report the mean value±SD. The observed differences between experimental conditions are shown as fold increase.

### Results

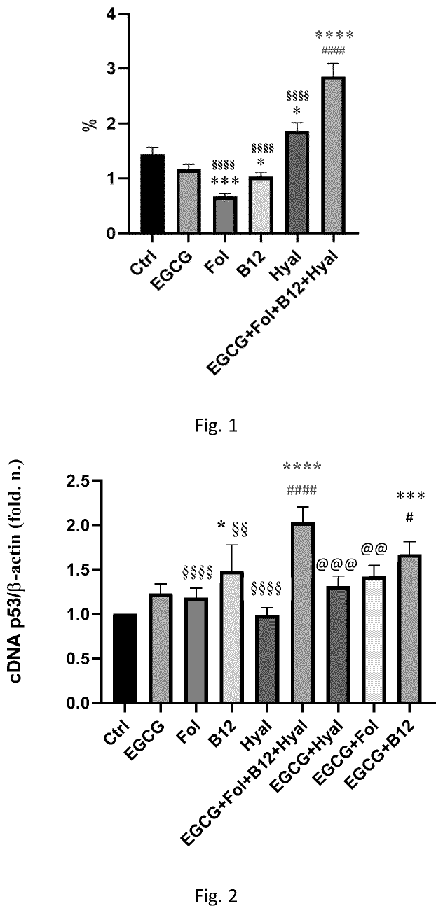

In Hela cervical cancer cells, the combination of EGCG, folic acid, vitamin B12, and hyaluronic acid synergistically increased cell death (apoptosis) compared to the control (****p<0.0001), EGCG (^{####}p<0.0001), Fol, B12, and Hyal alone (^{§§§§§} p<0.0001). In our cell model no differences in apoptosis levels between EGCG and the control group were observed. B12 and Hyal singularly administered significantly increased apoptosis compared to control (*p<0.05), nevertheless, this increase was not as significant as in the sample treated with the combination of all substances (****p<0.0001). Fol, alone, significantly decreased apoptosis compared with control (***p<0.001) (FIG. 1)

The association of EGCG, FA, B12 and HA modulates gene expression levels of p53 and E6/E7. After 48 hr of treatment, the combination of EGCG, Fol, B12 and Hyal synergistically increased the expression of p53 compared to control (****p<0.0001), EGCG alone (^{####}p<0.0001) and singular administration of Fol, Hyal, and B12 (^{$$$$}p<0.0001 and ^{§§} p<0.01). Aside from B12 which demonstrated a minor increase (*p<0.05), each listed substance when administered singularly did not increase p53 expression compared with control. The combination of EGCG, Fol, B12, and Hyal induced a significant increase of p53 compared to the combinations of EGCG+Hyal (^{@@@}p<0.001) and EGCG+Fol (^{@@}p<0.01). The differences between EGCG+Fol+B12+Hyal vs. EGCG+B12 were not significant, however, it is important to outline that, even if EGCG+B12 significantly increased p53 compared to the control (***p<0.001) and EGCG alone (^{#}p<0.05), the significance was less than those induced by the combination of EGCG+Fol+B12+Hyal (****p<0.0001) (FIG. 2).

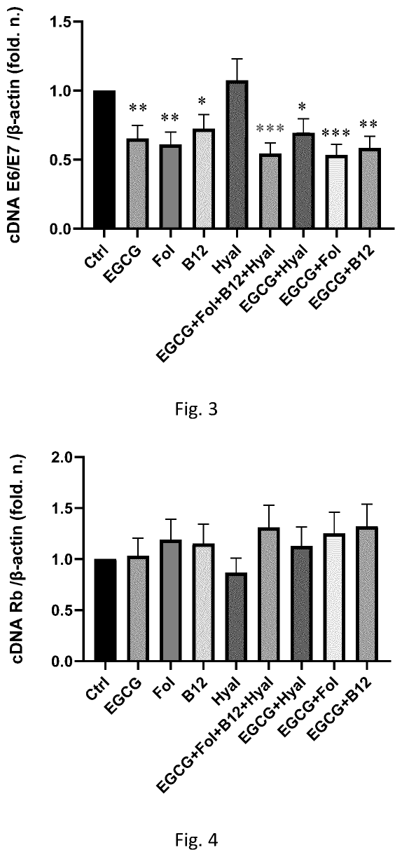

The combination of EGCG+Fol+B12+Hyal significantly and synergistically reduced the expression of E6/E7 (***p<0.001) which are partially responsible for tHPV persistence. In our experimental model, the combination of EGCG+Fol+B12+Hyal demonstrated the most significant decrease in E6/E7. (FIG. 3). Rb expression did not changed after a 48 hr treatment (FIG. 4).

### CONCLUSIONS

The invention demonstrates for the first time that the combination of EGCG, Fol, B12, and Hyal significantly and synergistically upregulated apoptosis in Hela cells, a model of persistence of the HPV infection. The combination of EGCG, Fol, B12, and Hyal increased apoptosis via expression of p53, known as the “the guardian of genome”, and inhibited by E6/E7 viral proteins partially responsible for HPV persistence.

Moreover, the combination of EGCG, Fol, B12, and Hyal significantly downmodulated the expression of E6/E7. No effects were on Rb expression.

1-8. (canceled)

9. A composition comprising a compound of formula (I): 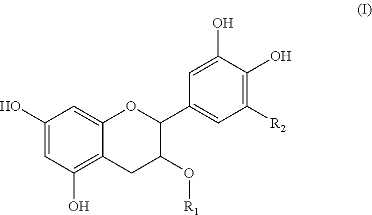
wherein R₁ is H or 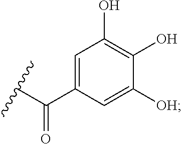 [SMILES: *C(=O)c1cc(O)c(O)c(O)c1]
and
R₂ is H or OH;
or stereoisomer or salt thereof;
and a further agent selected from the group consisting of:
a. folic acid or a salt or a derivative thereof;
b. vitamin B12 or a derivative thereof;
c. very low/low molecular weight hyaluronic acid or a pharmaceutically acceptable salt thereof; and
d. combinations thereof.

10. The composition of claim 9 comprising the compound of formula (I): 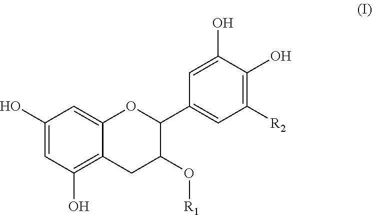
wherein R₁ is H or 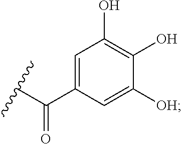 [SMILES: *C(=O)c1cc(O)c(O)c(O)c1]
and
R₂ is H or OH;
or stereoisomer or salt thereof;
and at least two further agents selected from:
a. folic acid or a salt or a derivative thereof;
b. vitamin B12 or a derivative thereof; and
c. very low/low molecular weight hyaluronic acid or a pharmaceutically acceptable salt thereof.

11. The composition according to claim 9 comprising i) the compound of formula (I) or stereoisomer or salt thereof and ii) folic acid or a salt or a derivative thereof iii) vitamin B12 or a derivative thereof and iv) very low/low molecular weight hyaluronic acid or a pharmaceutically acceptable salt thereof.

12. The composition according to claim 9 comprising:
a. the compound of formula (I) in an amount from 1 mg to 800 mg;
b. folic acid or a salt or a derivative thereof in an amount from 1 to 400 micrograms; and/or
c. vitamin B12 or a derivative thereof in an amount from 1 to 1000 micrograms; and/or
d. very low/low molecular weight hyaluronic acid or a pharmaceutically acceptable salt thereof in an amount from 1 mg to 200 mg.

13. The composition according to claim 9 wherein the compound of formula (I) is comprised in powdered leaves of a tea plant or in an extract thereof.

14-18. (canceled)

19. A method of the treatment, prevention, control and/or inhibition of human papilloma virus infection (HPV), comprising administering to a subject in need thereof a combination comprising a compound of formula (I): 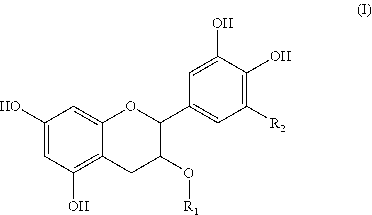
wherein R₁ is H or 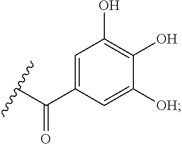 [SMILES: *C(=O)c1cc(O)c(O)c(O)c1]
and
R₂ is H or OH;
or stereoisomer or salt thereof;
and a further agent selected from the group consisting of:
a. folic acid or a salt or a derivative thereof;
b. vitamin B12 or a derivative thereof;
c. very low/low molecular weight hyaluronic acid or a pharmaceutically acceptable salt thereof; and
d. combinations thereof.

20. The method of claim 19, wherein the compound of formula (I) is selected from the group consisting of: (−)-epigallocatechin gallate (EGCG), (−)-gallocatechin gallate (GCG), (−)-epicatechin gallate (ECG), (−)-catechin gallate (CG), (−)-epicatechin (EC), (+)-catechin (C), (−)-epigallocatechin (EGC), (+)-gallocatechin (GC), and combinations thereof.

21. The method of claim 20, wherein the compound of formula (I) is (−)-epigallocatechin gallate (EGCG).

22. The method of claim 19, wherein:
a. the amount of the compound of formula (I) is from 1 mg to 800 mg per day;
b. the amount of folic acid or a salt or a derivative thereof is from 1 to 400 micrograms per day;
c. the amount of vitamin B12 or a derivative thereof is from 1 to 1000 micrograms per day; and
d. the amount of very low/low molecular weight hyaluronic acid or a pharmaceutically acceptable salt thereof is from 1 mg to 200 mg per day.

23. The method of claim 22, wherein:
a. the amount of the compound of formula (I) is from 50 mg to 600 mg per day;
b. the amount of folic acid or a salt or a derivative thereof is from 50 to 400 micrograms per day;
c. the amount of vitamin B12 or a derivative thereof is from 100 to 1000 micrograms per day; and
d. the amount of very low/low molecular weight hyaluronic acid or a pharmaceutically acceptable salt thereof is from 25 mg to 150 mg per day.

24. The method of claim 23, wherein:
a. the amount of the compound of formula (I) is about 200 mg per day;
b. the amount of folic acid or a salt or a derivative thereof is from 200 to 400 micrograms per day;
c. the amount of vitamin B12 or a derivative thereof is from 500 to 1000 micrograms (1 mg) per day; and
d. the amount of very low/low molecular weight hyaluronic acid or a pharmaceutically acceptable salt thereof is from 50 to 100 mg per day.

25. The method of claim 24, wherein:
a. the amount of the compound of formula (I) is about 200 mg per day;
b. the amount of folic acid or a salt or a derivative thereof is 400 micrograms per day;
c. the amount of vitamin B12 or a derivative thereof is 1000 micrograms (1 mg) per day; and
d. the amount of very low/low molecular weight hyaluronic acid or a pharmaceutically acceptable salt thereof is about 50 mg per day.

26. The method of claim 19, the compound of formula (I) or stereoisomer or salt thereof is administered with at least two of the further agents.

27. The method of claim 19, wherein the compound of formula (I) or stereoisomer or salt thereof is administered with a combination of folic acid or a salt or a derivative thereof, vitamin B12 or a derivative thereof and iv) very low/low molecular weight hyaluronic acid or a pharmaceutically acceptable salt thereof.

28. The method of claim 19 wherein the compound of formula (I) is comprised in powdered leaves of a tea plant or in an extract thereof.

29. The method of claim 28, wherein said leaves of a tea plant are green tea plant leaves.

30. The method of claim 29, wherein the green tea plant is Camellia Sinensis.

31. The method of claim 28, wherein said extract comprises EGCG in an amount of from about 30% by weight to about 95% by weight.

32. The method of claim 19, wherein the compound of formula (I) and the further agent are administered simultaneously and/or orally and/or topically once and/or twice a day.

33. The method of claim 19, further comprising a further therapeutic intervention, said therapeutic intervention being a surgery to remove human papilloma virus lesions.
